(19) United States
(12) Patent Application Publication
(10) Pub. No.: US 2005/0177880 A1
(43) Pub. Date: Aug. 11, 2005

(54) Nucleic acid isolated from the 5' nontranslated region (5'NTR) end of the human FGF-1 gene having IRES activity

(76) Inventors: Yvan Martineau, Toulouse (FR); Anne-Catherine Prats, Toulouse (FR); Christine Le Bec, Corbeil-Essonnes (FR)

Correspondence Address: HESLIN ROTHENBERG FARLEY & MESITI PC, 5 COLUMBIA CIRCLE, ALBANY, NY, 12203, US

(21) Appl. No.: 11/021,066

(22) Filed: Dec. 22, 2004

Related U.S. Application Data
(63) Continuation of application No. PCT/FR03/01969, filed on Jun. 26, 2003.

(30) Foreign Application Priority Data
Jun. 27, 2002 (FR) 02.08009

(51) Int. Cl. C12Q 1/68; A01K 67/00; C07H 21/04; C12N 9/22

(52) U.S. Cl. 800/8; 435/6; 435/69.1; 435/199; 435/320.1; 435/325; 536/23.2

(57) ABSTRACT

The invention relates to an isolated nucleic acid of the human FGF-1 gene having IRES (internal ribosome entry site) activity, said nucleotide sequence being derived from the 5′ non-translated region (5′NTR) end of the human FGF-1 gene. The invention also concerns an expression cassette of at least one molecule of interest in a eukaryotic cell comprising at least one of said sequences. The cassette can be inserted directly or in the form of a vector in a host cell, hence the invention further relates to the recombinant vectors and the host cell transfected by said vector or the cassette.

The invention relates to an isolated nucleic acid having IRES (internal ribosome entry site) activity, said nucleotide sequence originating from the 5′ nontranslated region (5′NTR) end of the human FGF-1 gene. The invention also relates to a cassette for expression of at least one molecule of interest in a eukaryotic cell, said cassette comprising the FGF-1 IRES and making it possible to coexpress several molecules from the same mRNA. The cassette can be introduced directly into host cells, or can be introduced in the form of a vector, such that the recombinant vectors and the host cells transfected with said vector or the cassette are also part of the invention.

The regulation of gene expression occurs at several levels: transcription, maturation, mRNA stability, and also translation. The translation of an mRNA is subject to strict regulation and proves to be a key step in the regulation of gene expression. For example, an increase in translation of the c-myc proto-oncogene is associated with Bloom's syndrome (20). This aberrant translational control could contribute to the increased risk that these patients run of developing cancers.

Regulation at the translational level involves many proteins and occurs mainly via the 5′ and 3′ nontranslated regions (NTRs), without excluding, however, certain structural elements of the coding portion of mRNAs. The translation initiation step is, itself also, subjected to regulation.

Conventional initiation of translation occurs through recognition of the cap located at the 5′ end of the transcript. This structure allows the translation initiation factors to bind. In this step, it is the eIF4F complex which plays the role of linker between the cap and the 40S small ribosomal subunit. This complex consists of three factors, respectively eIF4E (cap binding protein), eIF4A (an RNA helicase) and eIF4G which plays a bridging role, between the other translation initiation factors, the mRNA and the 40S small subunit. The latter, once recruited, will “scan” the mRNA until it encounters a (canonic or noncanonic) translation initiation codon, after which, the 60S subunit will associate with the 40S subunit and translation of the reading frame will begin until a stop codon is reached, or the ribosome dissociates.

Certain messenger mRNAs lack a cap and cannot therefore be translated via the mechanism described by Marylin Kozak. This is the case of picornaviral RNAs (14), where another system for initiating translation has been demonstrated, involving an internal ribosome entry site or IRES in the 5′ nontranslated region (5′NTR). This site enables the ribosomes to bind directly within the mRNA, via a mechanism that is still poorly understood. Since this discovery, IRESs have been identified in the RNA of other groups of viruses such as flaviviruses, pestiviruses, tobamoviruses and retroviruses (19).

The involvement of cellular proteins in the translation of these viral RNAs has prompted research on this mechanism of internal entry in transcripts of cellular origin. In 1991, such a site was characterized in the mRNA of BIP (immunoglobulin heavy-chain binding protein) (8) and, more recently, in that of the fibroblast growth factor FGF-2 (18), of the c-myc proto-oncogene (12), of the growth factor PDGF (platelet derived growth factor) (2) and of VEGF (vascular endothelial growth factor) (6).

The FGF-2 mRNA has a long 5′NTR of approximately 500 nucleotides in which alternative translation initiations have been demonstrated at noncanonic CUG codons (1), which result in the formation of five isoforms of FGF-2. An internal ribosome entry site has also been located between nucleotides 154 and 318 (data not published).

The VEGF mRNA itself also has a long nontranslated sequence in which the presence of an alternative initiation has, very recently, been demonstrated at a noncanonic CUG codon. In this 5′NTR, the presence of two IRESs has been demonstrated, one located upstream of the CUG and the other preceding the conventional AUG initiation codon (6).

Surprisingly, the production of these two growth factors is controlled, at the translational level, by IRESs which have activity in angiogenesis. These two factors are induced during the process of neovascularization of a developing tumor. There is a third major angiogenic factor, FGF-1, translation of the mRNA of which has not been studied to date.

Now, the applicants have discovered that the initiation of translation of FGF-1 mRNA involves an internal entry site located in the 5′ nontranslated region. In addition, they have noted that transcript A of FGF-1 in particular has, unexpectedly, an IRES activity in vivo (mouse muscle) that is better than the other cellular RNA IRESs tested up until now.

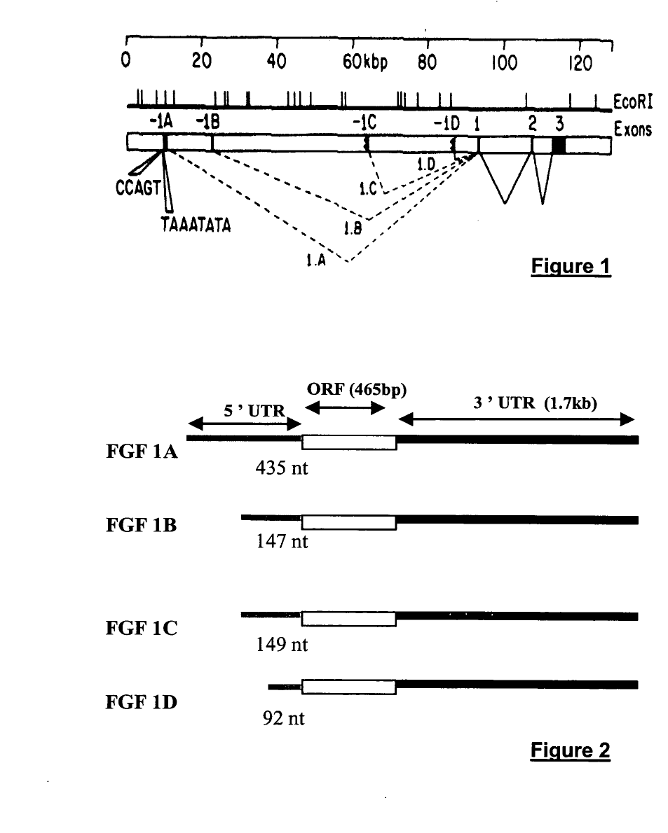

The genomic structure (11) of FGF-1 comprises three exons containing the coding sequence (FIG. 1). The latter undergo splicing which results in the formation of a single reading frame. Exon 3 contains the 3′NTR end which has a polyadenylation site.

Four different promoters exist for the human FGF-1 gene, which, once activated, result in the formation of four different primary transcripts. After maturation, the only difference between these four transcripts lies in their respective 5′NTR (FIG. 2). Exon 1 has a splice acceptor site where one of the four exons that is noncoding (A, B, C, D) attaches subsequent to the activation of their respective promoter.

Transcripts A and B are the major transcripts which are mainly expressed in the brain, the kidney and the retina. (FIG. 2). These two transcripts have 5′NTRs of 435 and 147 nucleotides, respectively. Transcripts C and D are minor transcripts expressed very weakly in vivo (11). However, they are present in glioblastoma-derived lines and can be induced in fibroblasts or smooth muscle cells of vascular origin when they are subjected to serum deprivation or treatment with PMA (phorbol 12-myristate 13-acetate). These two transcripts have 5′NTRs of 149 and 92 nucleotides, respectively.

The existence of these four mRNA variants encoding FGF-1 is common to humans, mice (9) and bovines (16). Comparison of the sequences of these human 5′NTRs has shown good homology with those from bovines and from mice (9). In fact, alignment of the sequences of the FGF-1 5′NTRs from these various organisms (9, 11, 16) has shown good conservation of these noncoding sequences, with close to 70% similarity for the 5′NTR A. Such a conservation in the sequences of nontranslated regions makes it possible to envision an important function for these 5′NTRs, regions that are usually subjected to relatively low selection pressure and therefore to rapid evolution.

The IRES activity of the 5′NTRs of each of the transcripts A, B, C and D of human and murine FGF-1 is demonstrated in the subsequent description (example 1, B1).

Mostly, the presence of IRES is sought by transfecting Cos-7 cells with CAT-I-NuCAT vectors containing the FGF-1 5′NTRs and then subjecting the transfected cells to western blotting. Expression of the NuCAT protein is observed in the presence of the transcripts A, B, C and D and reflects the IRES activity of these four transcripts (qualitative analysis). Due to the strong homology of the human 5′NTR sequences with those from bovines and in particular from mice (9), it is deduced therefrom that IRES sites are also present in murine and bovine FGF-1.

The in vitro IRES activity of the 5′NTRs of each of the human FGF-1 transcripts is also studied as a function of the cell type and state (quantitative analysis) (example 1 B1).

To do this, the IRES activity is measured by means of bicistronic vectors encoding reporter genes (in practice, the LucF/LucR genes) in various lines. Calculating the LucF/LucR ratio provides information regarding the internal entry capacity of the 5′NTR sequence and defines the IRES activity of this sequence. Among the various results, very good IRES activity of the transcript A is observed in Hela and Sk-Hep-1 cells, and is greater than that of the FGF-2 IRES.

The applicants have, moreover, compared the IRES activity of the structure of the human 5′NTRs (transcripts A and B) with that of the murine 5′NTRs, in vitro, on human cells (example 1 B2). The transfections were carried out in Cos-7, Sk-Hep-1 and Saos-2 cells in order to perform a direct comparison with the human sequences. The experiments showed that the IRES activity profiles for the human and murine A and B variants were similar.

The invention consists not only in having demonstrated, for the first time, the IRES activity of the 5′NTRs of FGF-1, but also, as will subsequently be demonstrated, in having characterized the IRES sites in the various transcripts A, B, C and D of human FGF-1 (example 2). More particularly, the applicant has succeeded in identifying the smallest sequence of each of the transcripts A, B, C and D which has IRES activity, both in vitro and in vivo. To do this, various continuous nucleotide sequences derived from the FGF-1 messenger RNA were subcloned into a biocistronic vector encoding luciferase reporter genes. Various mammalian and human cell lines (in vitro) were transfected, and also mouse muscle (in vivo) was transduced with these vectors.

The IRES activity of human FGF-1 and, by homology, of murine FGF-1 is therefore disclosed for the first time in the present application. This activity has been demonstrated for each transcript A, B, C and D, as it has to certain fragments of the 5′NTRs of these transcripts.

In the remainder of the description and in the claims, the expression “homologous sequence” denotes a sequence exhibiting at least 60% identity, advantageously 80%, preferably 95%, with a given sequence, the homology resulting, in this case, from the variability from one species to another, in particular from mouse to human.

Consequently, the invention relates, first of all, to the use, for its IRES activity, of an isolated nucleic acid containing a sequence identical or homologous to the sequence SEQ ID 1 located between nucleotides 1 and 435 of the 5′NTR of the transcript A of the human FGF-1 mRNA, and hereinafter referred to as A. This sequence corresponds to the entire sequence of the 5′NTR of the transcript A.

As already mentioned, the applicant has also succeeded in identifying, on the transcript A, nucleotide sequences that are shorter than the sequence SEQ ID 1, and have IRES activity, which therefore suggests that the IRES site is included in these sequences.

Thus, a subject of the invention is also, in particular for use for its IRES activity:
an isolated nucleic acid having IRES activity, comprising a sequence identical to the sequence SEQ ID 2, located between nucleotides 1 and 390 of the sequence SEQ ID 1 and hereinafter referred to as A2,
an isolated nucleic acid having IRES activity, comprising a sequence identical or homologous to the sequence SEQ ID 3, located between nucleotides 39 and 435 of the sequence SEQ ID 1 and hereinafter referred to as A3,
an isolated nucleic acid having IRES activity, comprising a sequence identical or homologous to the sequence SEQ ID 4, located between nucleotides 122 and 435 of the sequence SEQ ID 1 and hereinafter referred to as A4,
an isolated nucleic acid having IRES activity, comprising a sequence identical or homologous to the sequence SEQ ID 5, located between nucleotides 214 and 435 of the sequence SEQ ID 1 and hereinafter referred to as A5,
an isolated nucleic acid having IRES activity, comprising a sequence identical or homologous to the sequence SEQ ID 6, located between nucleotides 122 and 390 of the sequence SEQ ID 1 and hereinafter referred to as A6,
an isolated nucleic acid having IRES activity, comprising a sequence identical or homologous to the sequence SEQ ID 7, located between nucleotides 214 and 390 of the sequence SEQ ID 1 and hereinafter referred to as A7,
an isolated nucleic acid having IRES activity, comprising a sequence identical or homologous to the sequence SEQ ID 8, located between nucleotides 269 and 435 of the sequence SEQ ID 1 and hereinafter referred to as A8,
an isolated nucleic acid having IRES activity, comprising a sequence identical or homologous to the sequence SEQ ID 9, located between nucleotides 269 and 390 of the sequence SEQ ID 1 and hereinafter referred to as A9,
an isolated nucleic acid having IRES activity, comprising a sequence identical or homologous to the sequence SEQ ID 12, located between nucleotides 1 and 366. of the sequence SEQ ID 1 and hereinafter referred to as A12,
an isolated nucleic acid having IRES activity, comprising a sequence identical or homologous to the sequence SEQ ID 16, located between nucleotides 122 and 366 of the sequence SEQ ID 1 and hereinafter referred to as A16,
an isolated nucleic acid having IRES activity, comprising a sequence identical or homologous to the sequence SEQ ID 17, located between nucleotides 214 and 366 of the sequence SEQ ID 1 and hereinafter referred to as A17,
an isolated nucleic acid having IRES activity, comprising a sequence identical or homologous to the sequence SEQ ID 18, located between nucleotides 269 and 366 of the sequence SEQ ID 1 and hereinafter referred to as A18,
an isolated nucleic acid having IRES activity, comprising a sequence identical or homologous to the sequence SEQ ID 19, located between nucleotides 1 and 344 of the sequence SEQ ID 1 and hereinafter referred to as A19,
an isolated nucleic acid having IRES activity, comprising a sequence identical or homologous to the sequence SEQ ID 20, located between nucleotides 1 and 330 of the sequence SEQ ID 1 and hereinafter referred to as A20.

The nucleic acid corresponding to the sequence SEQ ID 18 (A18) constitutes the shortest human FGF-1 transcript A fragment having IRES activity. For a sequence which is shorter, of the order of 100 nucleotides, no significant IRES activity is obtained, at least in human adenocarcinoma cells. In fact, and as emerges from the examples hereinafter, the IRES activity is not significant for the following fragments:
sequence identical or homologous to the sequence SEQ ID 10, located between nucleotides 303 and 435 of the sequence SEQ ID 1 and hereinafter referred to as A10,
sequence identical or homologous to the sequence SEQ ID 11, located between nucleotides 303 and 390 of the sequence SEQ ID 1 and hereinafter referred to as A11,
sequence identical or homologous to the sequence SEQ ID 13, located between nucleotides 282. and 435 of the sequence SEQ ID 1 and hereinafter referred to as A13,
sequence identical or homologous to the sequence SEQ ID 14, located between nucleotides 282 and 390 of the sequence SEQ. ID 1 and hereinafter referred to as A14,
sequence identical or homologous to the sequence SEQ ID 15, located between nucleotides 282 and 366 of the sequence SEQ ID 1 and hereinafter referred to as A15.

As already mentioned, the applicant has demonstrated that the 5′NTR end of the human FGF-1 transcript B has considerable IRES activity, in particular in myoblasts.

Consequently, the invention also relates to an isolated nucleic acid having IRES activity, which is characterized in that it contains a sequence identical or homologous to the sequence SEQ ID 30, located between nucleotides 1 and 147 of the 5′NTR of the transcript B of the human FGF-1 mRNA, and hereinafter referred to as B. In other words, the sequence SEQ ID 30 corresponds to the entire sequence of the 5′NTR of the transcript B.

The applicant has also succeeded in identifying, on the transcript B, nucleotide sequences that are shorter than the sequence SEQ ID 30, and have IRES activity, which therefore suggests that the IRES site is included in these sequences.

Thus, a subject of the invention is also:
an isolated nucleic acid having IRES activity, comprising a sequence identical to the sequence SEQ ID 31, located between nucleotides 1 and 93 of the sequence SEQ ID 30 and hereinafter referred to as B2.

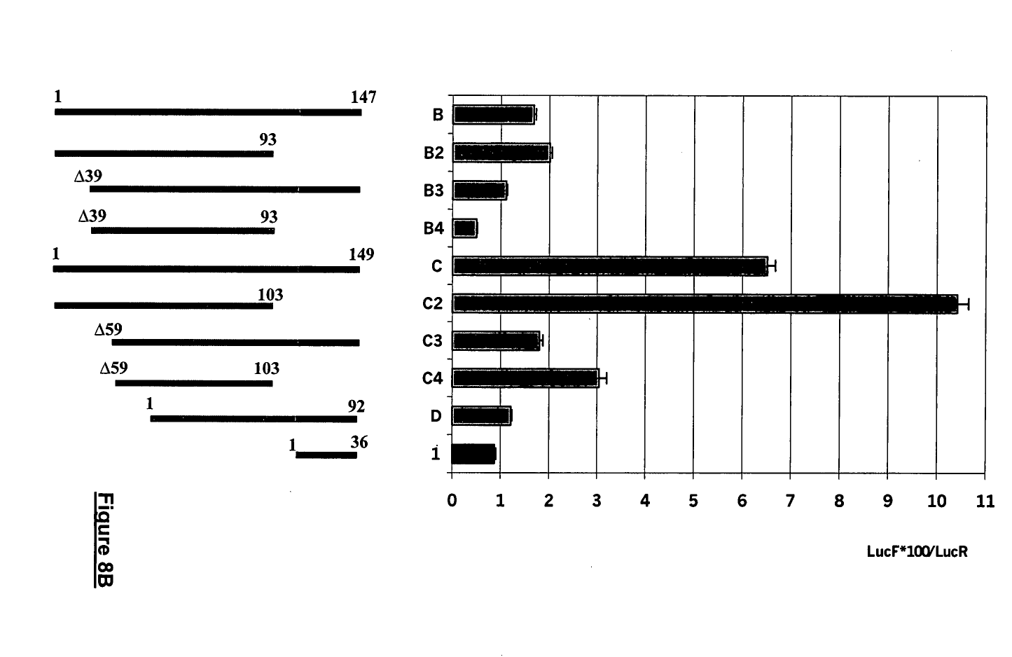

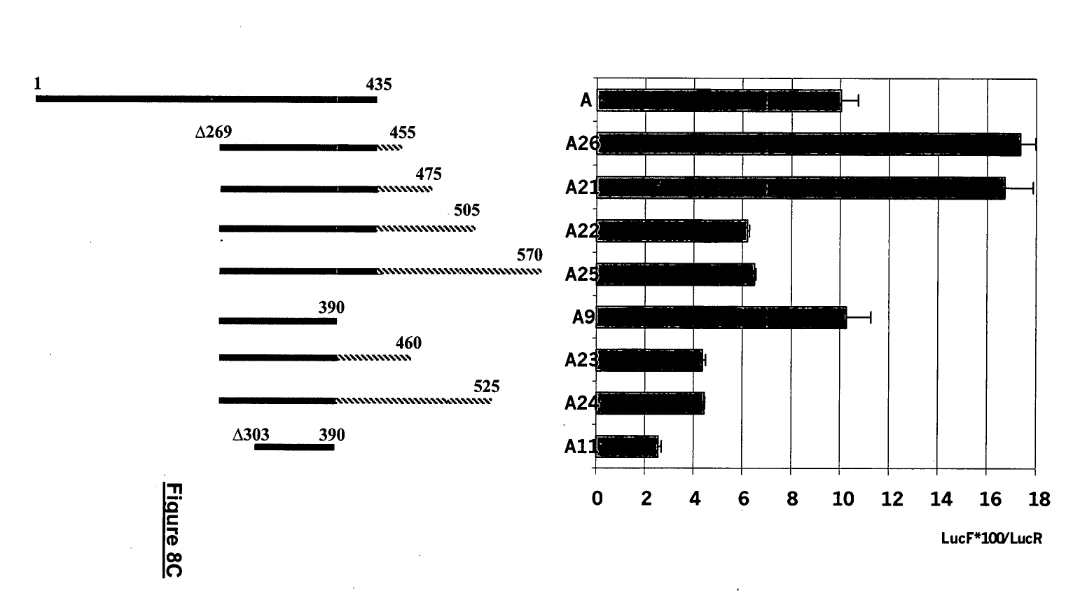

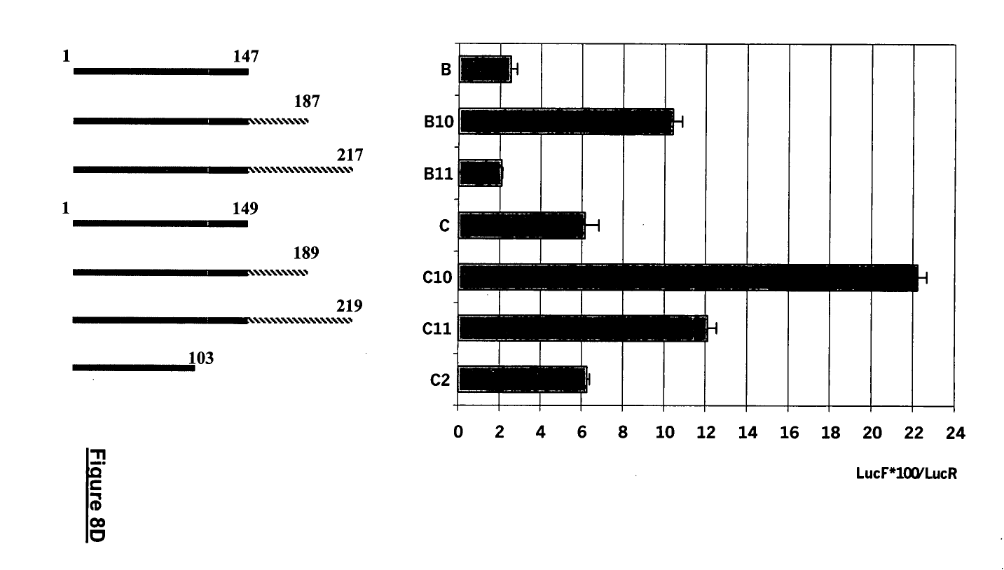

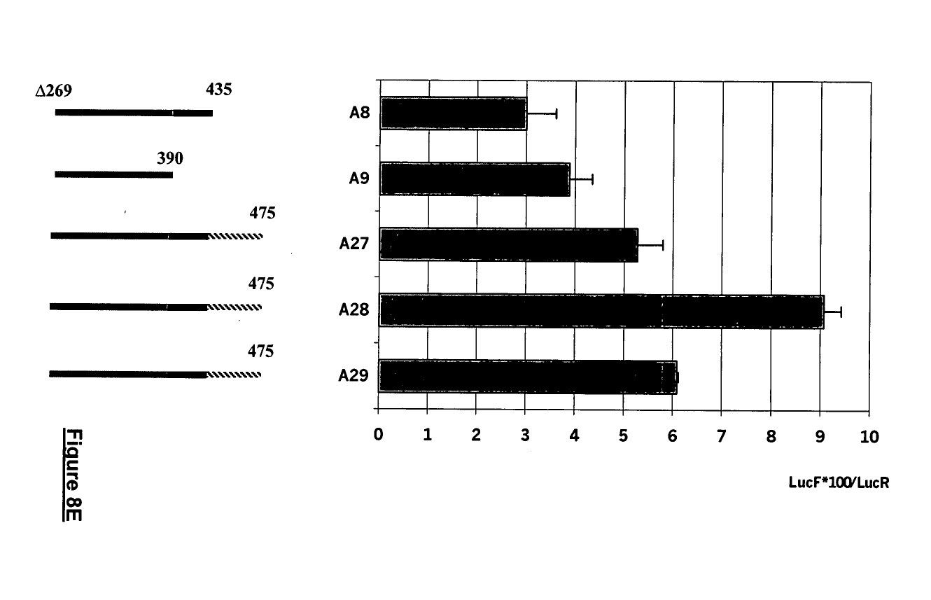

The nucleic acid corresponding to the sequence SEQ ID 31 (B2) constitutes the shortest human FGF-1 transcript B fragment having IRES activity. For a sequence from which the first 39 nt are deleted, no significant IRES activity is obtained, at least in human adenocarcinoma cells. In fact, and as emerges from the examples hereinafter (FIG. 8B), the IRES activity is not significant for the following fragment:
sequence identical or homologous to the sequence SEQ ID 32, located between nucleotides 39 and 147 of the sequence SEQ ID 30 and hereinafter referred to as B3,
sequence identical or homologous to the sequence SEQ ID 33, located between nucleotides 39 and 93 of the sequence SEQ ID 30 and hereinafter referred to as B4.

The applicant has also demonstrated that the 5′NTR end of the transcript C of human FGF-1 and one of its subfragments have considerable IRES activity.

Consequently, a subject of the invention is also an isolated nucleic acid having IRES activity, which is characterized in that it contains a sequence identical or homologous to the sequence SEQ ID 36, located between nucleotides 1 and 149 of the 5′NTR of the transcript C of the human FGF-1 mRNA, and hereinafter referred to as C. This sequence corresponds to the entire sequence of the 5′NTR of the transcript C.

Since the IRES site has been identified by the applicant between nucleotides 1 and 103 of the sequence SEQ ID 36, the invention also relates to an isolated nucleic acid having IRES activity, which is characterized in that it contains a sequence identical or homologous to the sequence SEQ ID 37, located between nucleotides 1 and 103 of the sequence SEQ ID 36, hereinafter referred to as C2.

For a sequence from which the first 59 nt are deleted, no significant IRES activity is obtained, at least in human adenocarcinoma cells. In fact, and as emerges from the examples hereinafter (FIG. 8B), the IRES activity is not significant for the following fragment:
an isolated nucleic acid exhibiting IRES activity, comprising a sequence identical to the sequence SEQ ID 38, located between nucleotides 59 and 149 of the sequence SEQ ID 36 and hereinafter referred to as C3,
an isolated nucleic acid exhibiting IRES activity, comprising a sequence identical to the sequence SEQ ID 39, located between nucleotides 59 and 103 of the sequence SEQ ID 36 and hereinafter referred to as C4.

Moreover, the applicant has demonstrated that the 5′NTR end of the transcript D of human FGF-1 has significant IRES acitivity, in particular on myoblasts.

Consequently, the invention also relates to an isolated or recombinant nucleic acid having IRES activity, which is characterized in that it contains a sequence identical or homologous to the sequence SEQ ID 42, located between nucleotides 1 and 92 of the 5′NTR of the transcript D of the human FGF-1 mRNA, and hereinafter referred to as D. The sequence SEQ ID 42 corresponds to the entire sequence of the 5NTR of the transcript D.

The applicants have, moreover, demonstrated that the IRES activity of certain fragments may be increased by adding a certain number of nucleotides positioned 3′ of the IRES.

In particular, the invention covers:
an isolated nucleic acid having IRES activity, containing a sequence identical or homologous to the sequence SEQ ID 26 (A26),

an isolated nucleic acid having IRES activity, containing a sequence identical or homologous to the sequence SEQ ID 21 (A21),
an isolated nucleic acid having IRES activity, containing a sequence identical or homologous to the sequence SEQ ID 34 (B10),
an isolated nucleic acid having IRES activity, containing a sequence identical or homologous to the sequence SEQ ID 40 (C10),
an isolated nucleic acid having IRES activity, containing a sequence identical or homologous to the sequence SEQ ID 41 (C11).

The IRES activity of human FGF-1 and murine FGF-1 make them potential candidates for the production of cassettes for expressing one or more genes of interest in eukaryotic cells, from the same mRNA under the control of a single promoter.

In the remainder of the description and in the claims, the expression “gene of interest” denotes nucleic acids encoding polypeptides such as proteins of therapeutic interest, antigenic proteins, anti-angiogenic proteins, pro-angiogenic proteins or tumor suppressor proteins, or else genes intended to compensate for a genetic anomaly such as a mutation, etc.

Consequently, and according to another aspect, the invention relates to a cassette for expressing at least one gene of interest in a eukaryotic cell, said cassette being characterized in that it contains at least one nucleic acid as described above.

As will be seen subsequently, the cassettes comprising a single gene may be used either for producing molecules of interest in vitro, or in gene therapy.

It is also possible to create a bicistronic cassette resulting from the insertion of one of the above-mentioned nucleic acid sequences between two genes of interest, the expression of the second gene being coordinated with that of the first gene. More precisely, the first gene (first cistron) provides the level of cap-dependent translation, while the second gene (second cistron) is expressed proportionally to the activity of internal ribosome entry in the various FGF-1 5′NTRs.

This principle can be extended to the coexpression of 3, 4 or more molecules insofar as they are separated by an IRES sequence as defined above, resulting in the development of multicistronic cassettes. Of course, the multicistronic cassette of the invention comprises FGF-1 IRESs as characterized in the present application, which may or may not be associated with other IRESs such as, for example, those identified on BIP (immunoglobulin heavy-chain binding protein), the fibroblast growth factor FGF-2, the c-myc proto-oncogene, the growth factor PDGF (platelet derived growth factor), and VEGF (vascular endothelial growth factor).

Consequently, and in a particular embodiment, the expression cassette contains at least two genes of interest separated by a nucleic acid as described above, the first gene (first cistron) providing the level of cap-dependent translation, while the second gene (second cistron) is expressed proportionally to the activity of the IRES site present in said nucleic acid.

In a known manner, the cassette also contains a promoter for controlling the expression of the gene(s).

The main application of the multicistronic expression cassettes comes from gene therapy, insofar as such cassettes make it possible to express the product of the nucleotide(s) or the genes that-they contain, from the same mRNA in vivo. These cassettes can also-make it possible to express various types of molecules of industrial interest, such as receptors, enzymes, etc. Gene therapy can be envisioned according to two distinct techniques, entirely known to those skilled in the art.

The first technique consists in introducing, in vivo, directly into the host organism (human or animal), a pharmaceutical composition containing the naked cassette contained in a double-standard DNA, said pharmaceutical composition being part of the present invention.

In the case of the use of the naked cassette, said cassette is coupled, in the pharmaceutical composition, to any compound capable of promoting the penetration of the cassette into cells, in particular PEI and its derivatives, liposomes and derivatives, etc.

The recombinant vectors contained in the pharmaceutical composition, into which the expression cassettes are inserted, are also part of the invention. Thus, the expression cassettes can be inserted into plasmid vectors or viral vectors such as retroviruses, lentiviruses, adenoviruses and AAVs.

The second gene therapy technique consists in removing a cell from the host organism, and then in introducing, in vitro, the naked cassette, or the cassette inserted into a vector, into the cell, the modified cell then being reintroduced into the host organism. In this way, the product of the cassette can be expressed in the patient's body.

As already mentioned, when the expression cassette contains only one molecule of interest, this cassette can be used for producing said molecule from cell cultures, in vitro. As previously, the cassette may be in naked form or in a form inserted into a vector. The cassette or the vector is then introduced into the host cell by means of a transfection technique well known to those skilled in the art. Such techniques may, for example, be physical techniques (electroporation), chemical techniques, synthetic vectors, calcium-phosphate precipitation, or else other methods such as cell fusion, conjugation, etc.

Consequently, the invention also relates to a recombinant host cell, which is characterized in that it comprises the expression cassette or a recombinant vector as described above.

The recombinant host cells of the invention are in particular, but without implied limitation:

| | | |
|---|---|---|
| 911 | human embryonic retinoblast cells, | Généthon line |
| | no ATCC No. | |
| C2/7 | murine myoblast cells; no ATCC No. | Généthon line |
| CHQ5B | human embryonic myoblast cells | |
| C2C12 | murine myoblast-derived line | 91031101 |
| Cos-7 | CV-1 transformed with the SV-40 | CRL-1654 |
| | virus T antigen | |
| Hela | human uterine carcinoma epithelial | CCL-2 |
| | cells | |
| L929 | immunodepressed mouse connective | CCL-1 |
| | tissues | |
| NIH-3T3 | murine embryonic fibroblast cells | CRL1658 |
| Saos-2 | human osteosarcoma epithelial cells | HTB-85 |
| | (p53 −/−) | |
| SK-Hep-1 | human liver adenocarcinoma | HTB-52 |
| SK-N-AS | human neuroblastoma cells | 94092302 |
| SK-N-BE | human neuroblastoma cells | 95011815 |

In practice, production of the peptide is obtained by transfection, in vitro, of the host cell placed in culture, with the naked cassette or cassette inserted into a vector.

Analysis of the expression of the peptide is carried out using cell extracts prepared from the transfected cells. According to the nature of the peptide of interest, its level of expression will be analyzed by various detection techniques (enzymatic assay, immunodetection, etc.). For the purpose of industrial use of the molecules of interest, they may be purified. For other uses, the cells genetically modified with the expression cassette will be used to produce the molecules of interest (ex vivo gene therapy, pharmacological screening).

The expression cassette produced from the IRES sequences which are the subject of the present invention can also be inserted into the cells (egg) of an animal in order to create a transgenic animal.

Consequently, the invention also relates to a transgenic animal, which is characterized in that its cells comprise the naked cassette or cassette inserted into a vector, as described above. In practice, the animal is obtained by introducing the cassette or the vector into the cells at the embryonic stage. This type of technology is entirely known to those skilled in the art.

The invention emerges clearly from the examples hereinafter, in support of the attached figures.

FIG. 1: The fibroblast growth factor 1 gene: Organization of the human FGF-1 gene. Exons 1, 2 and 3 constitute the 3 coding exons; splicing of the noncoding exons—1A, 1B, 1C and 1D, at exon 1 will generate the A, B, C and D mRNAs, respectively. The starting point for transcription of the transcript A is preceded by the canonic sequences CCAAT and TATA.

FIG. 2: Representation of the 4 transcripts A, B, C and D encoding FGF-1: the transcripts A and B are major transcripts and are expressed in the kidney, the brain and the retina. The transcripts C and D are minor transcripts and are expressed in fibroblasts and vascular smooth muscle cells (VSMCs) after induction with serum or with PMA. They are also expressed in cells derived from prostate cancer (PC3) or lung cancer, or cells derived from glioblastomas.

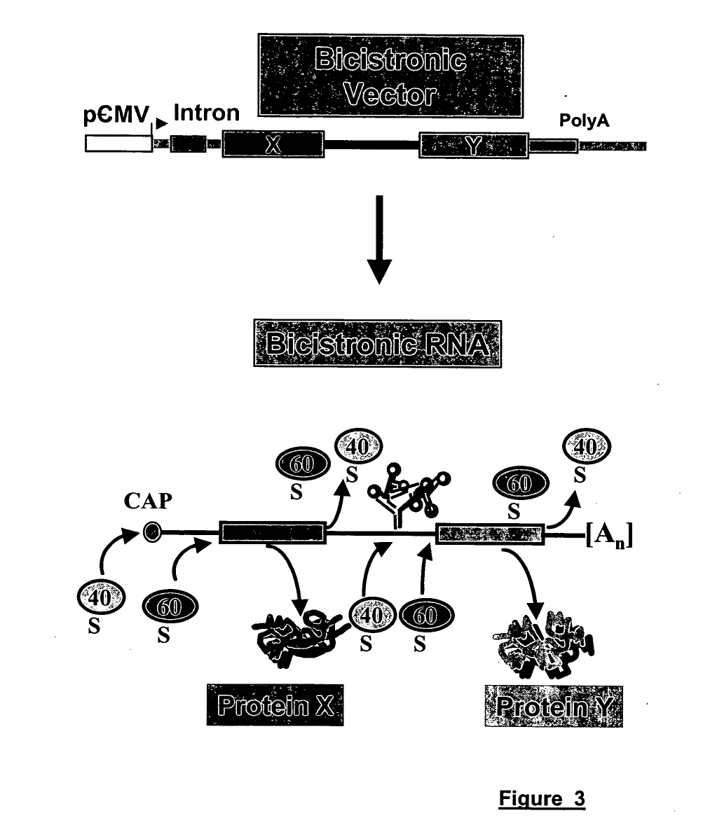

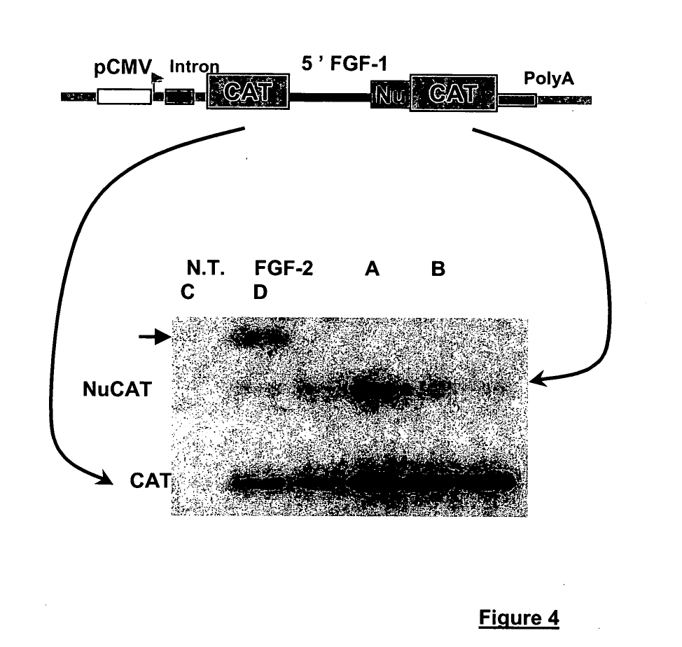

FIG. 3: IRES study strategy: Principle of the bicistronic vectors: the first cistron of the bicistronic mRNA provides the level of cap-dependent translation, the second is expressed proportionally to the activity of internal entry in the FGF-1 5′NTRs. The calculation of the protein Y/protein X ratio defines the IRES activity of the sequence located between the two cistrons.

FIG. 4: Western blotting subsequent to transfection of Cos-7 cells with the vectors CAT-I-NuCAT: the intensity of the bands corresponding to CAT provides the level of cap-dependent translation. The intensity of the NuCAT bands corresponds to the IRES activity of the 5′NTR under consideration. N.T.: extract from non-transfected cells; FGF-2: vector containing the FGF-2 5′NTR. A, B, C, D: vectors containing, respectively, the A, B, C and D 5′NTRs of human FGF-1; arrow 1: high molecular weight isoforms derived from the alternative translation initiation in the FGF-2 5′NTR at initiation codon CUG.

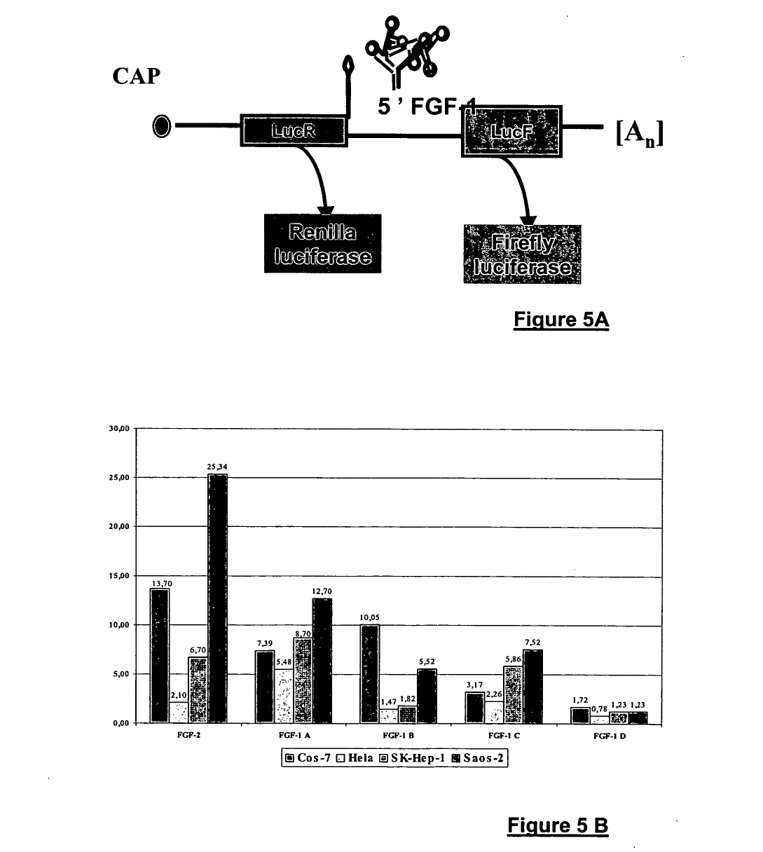

FIG. 5: Bicistronic LucR-I-LucF mRNAs and comparison of the IRES activity of the 5′NTRs of human FGF-1 in various cell types.

FIG. 5A: Diagrammatic representation of the LucR-I-LucF bicistronic mRNA. A stem-loop structure was placed in a position 3′ of LucR in order to limit the phenomenon of nonspecific translation reinitiation in the LucF second cistron.

FIG. 5B: Diagram of the IRES activities of the 5′NTRs of human FGF-1 in various cell types. The values along the Y-axis are obtained by calculating the ratio of the LucF/LucR activities and are expressed in arbitrary units.

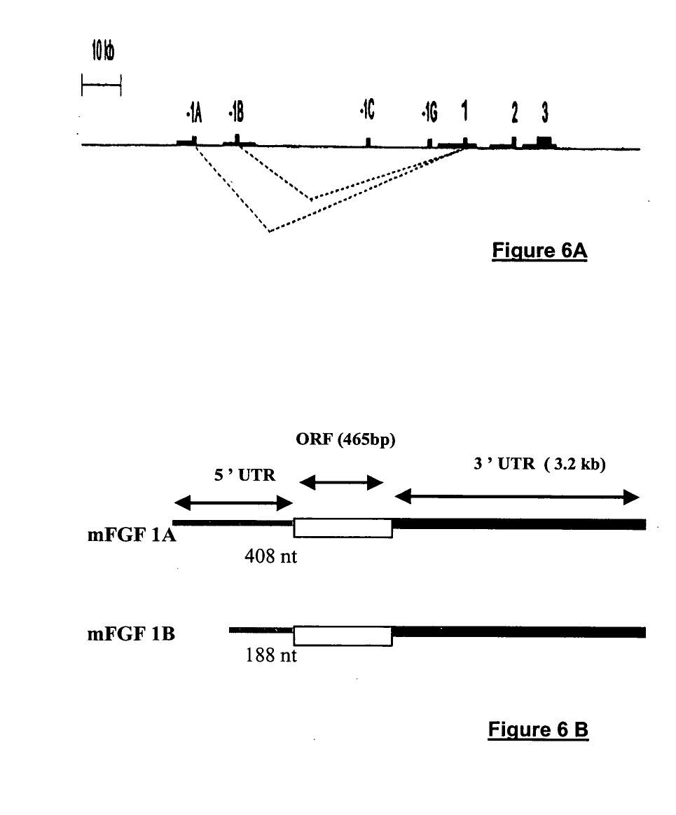

FIG. 6: Representation of the murine FGF-1 gene and transcripts.

FIG. 6A: Murine FGF-1 gene structure. Exons 1, 2 and 3 constitute the 3 coding exons; splicing of the noncoding exons −1A and −1B at exon 1 generates the A and B mRNAs of FGF-1.

FIG. 6B: Diagram of the murine FGF-1 transcripts A and B. The transcripts have the reading frame and the 3′NTR region in common, and differ by virtue of their 5′NTR end.

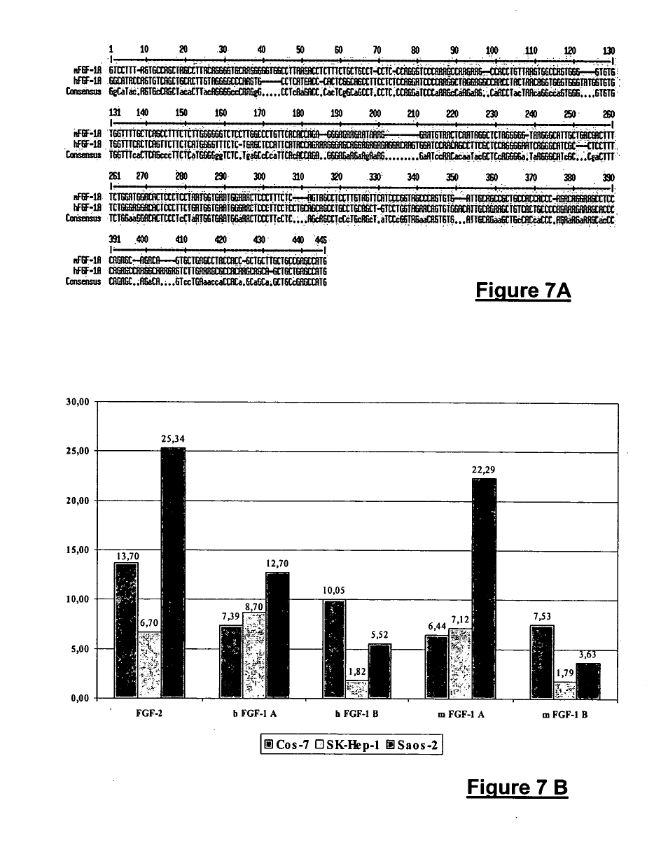

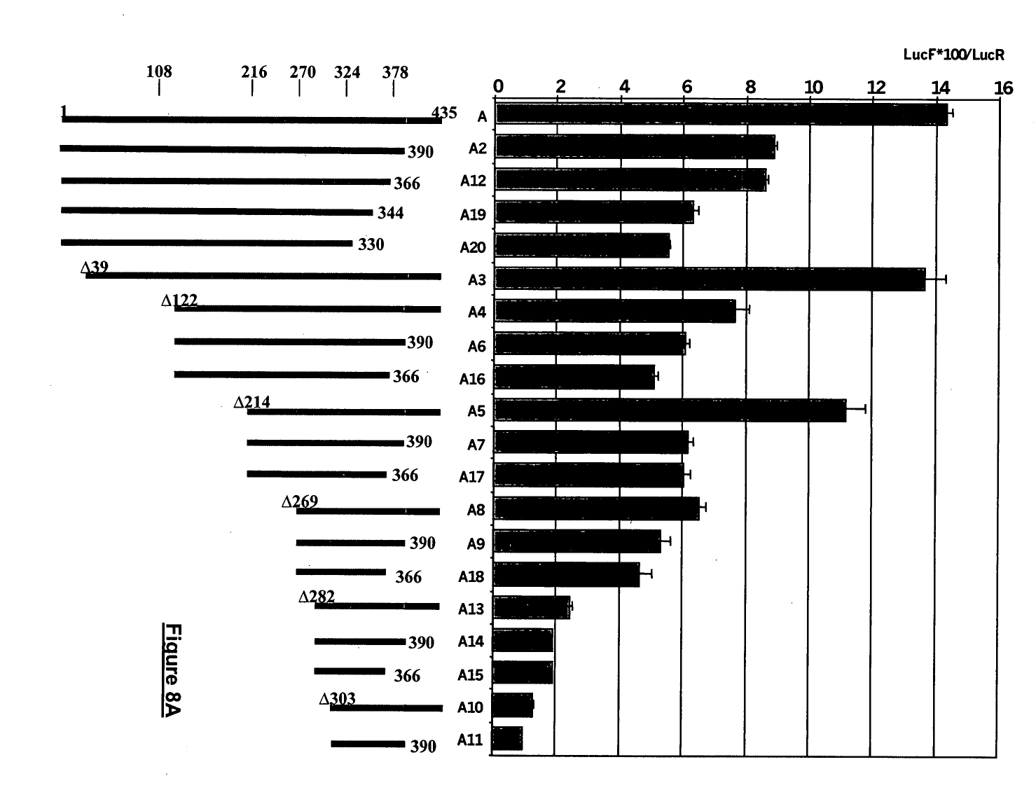

FIG. 7: Comparison between the human and murine 5′NTRs of FGF-1.

FIG. 7A: Alignment of the sequences of the 5′NTRs A of murine FGF-1 (mFGF-1) and human FGF-1 (hFGF-1).

FIG. 7B: Diagram of the IRES activities of the 5′NTRs A and B of human FGF-1 (hFGF1) and murine FGF-1 (mFGF1) in various cell types. The values along the Y-axis are obtained by calculating the ratio of the LucF/LucR activities and are expressed in arbitrary units.

FIG. 8: Diagram of the IRES activities of various human FGF-1 5′NTR subfragments in SK-Hep-1 human adenocarcinoma cells. The values along the Y-axis are obtained by calculating the ratio of the LucF/LucR activities and are expressed in arbitrary units.

FIG. 8A: IRES activity of 20 nucleotide sequences of fragment A (A, A2, A3, A4, A5, A6, A7, A8, A9, A10, A11, A12, A13, A14, A15, A16, A17, A18, A19, A20).

FIG. 8B: IRES activity of 4 nucleotide sequences of fragments B and C, respectively (B, B2, B3, B4) and (C, C2, C3, C4), and the nucleotide sequence of fragment D.

FIG. 8C: IRES activity of 6 mutated fragments A (A26, A21, A22, A23, A24, A25) in 911 (embryonic retinoblast) lines.

FIG. 8D: IRES activity of 2 mutated fragments B (B10, B11) and of 2 mutated fragments C (C10, C11) in 911 (embryonic retinoblast) lines.

FIG. 8E: IRES activity of 3 mutated fragments A27, A28, A29 in 911 (embryonic retinoblast) lines.

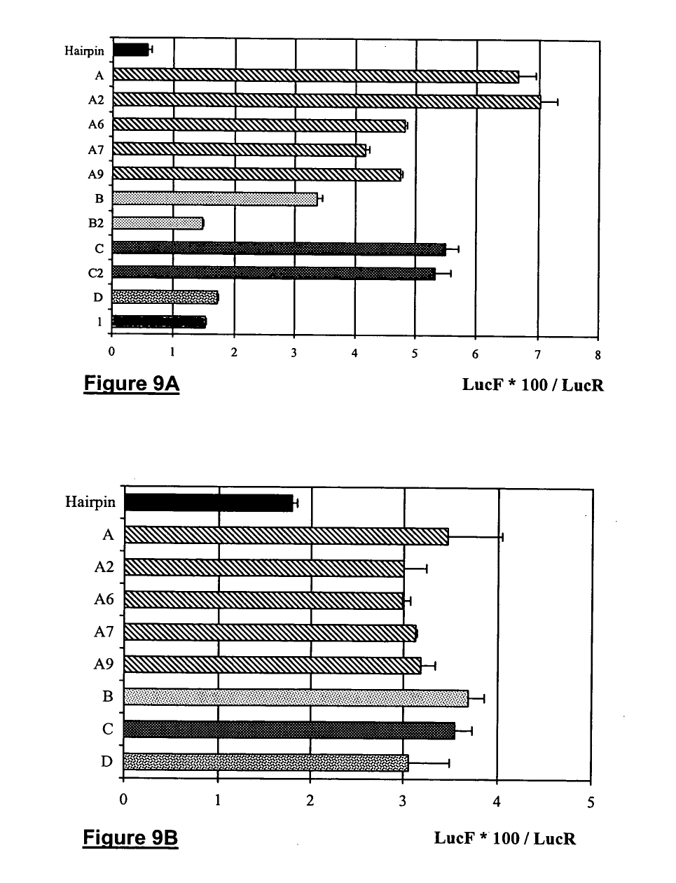

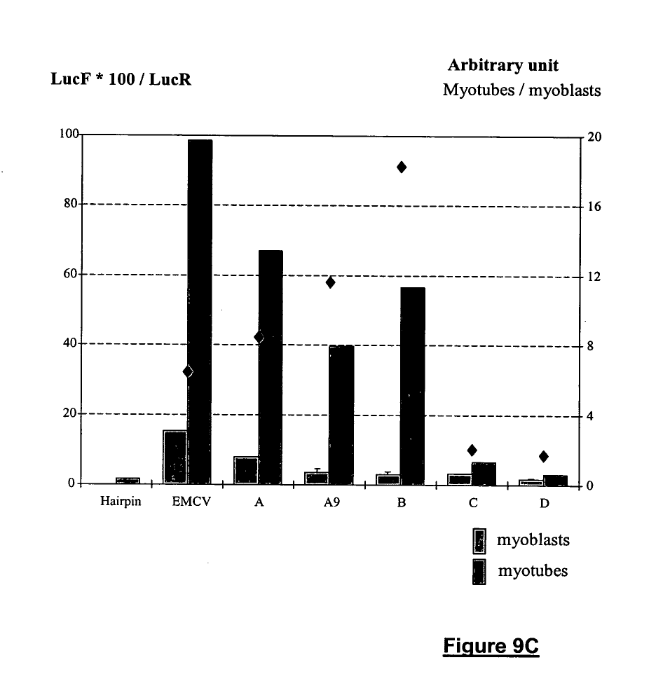

FIG. 9: Diagram of the IRES activities of various human FGF-1 5′NTR subfragments in 911 lines (embryonic retinoblasts (FIG. 9A)), myoblasts (FIG. 9B) and myoblasts differentiated into myotubes (FIG. 9C). The values along the Y-axis are obtained by calculating the ratio of the LucF/LucR activities and are expressed in arbitrary units. 5 nucleotide sequences of fragment A (A, A2, A6, A7, A9), the nucleotide sequence of fragment B, including that of fragment B2 for FIG. 9A, the nucleotide sequence of fragment C, including that of fragment C2 for FIG. 9A, and the nucleotide sequence of fragment D were also tested.

FIG. 10: Diagram comparing the IRES activity of the transcript A of human FGF-1 in vivo (intramuscular injection in mice), with regard to the IRES of EMCV, VEGF, FGF2, Bip and PDGF.

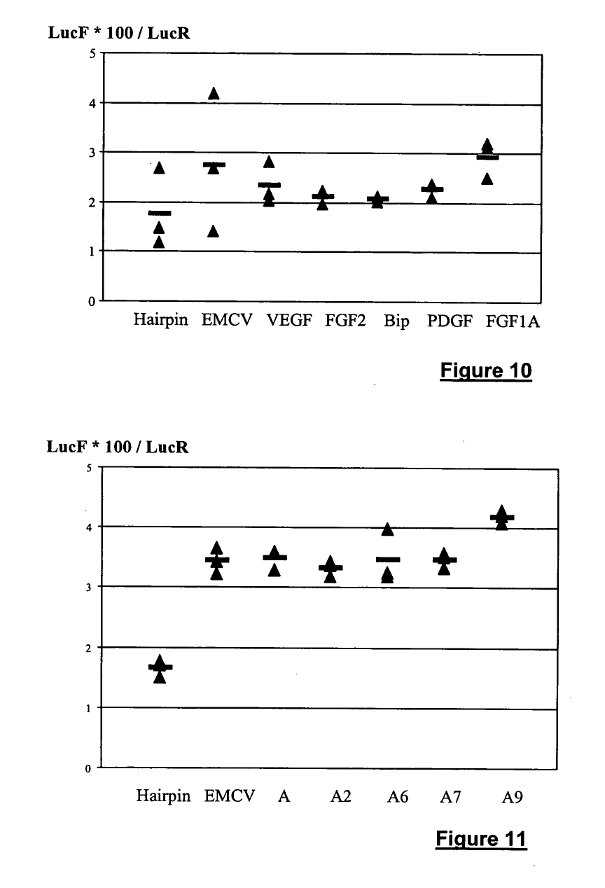

FIG. 11: Diagram comparing the IRES activity of the transcript A of human FGF-1 and of the subfragments A2, A6, A7 and A9, in vivo, with regard to the IRES of EMCV.

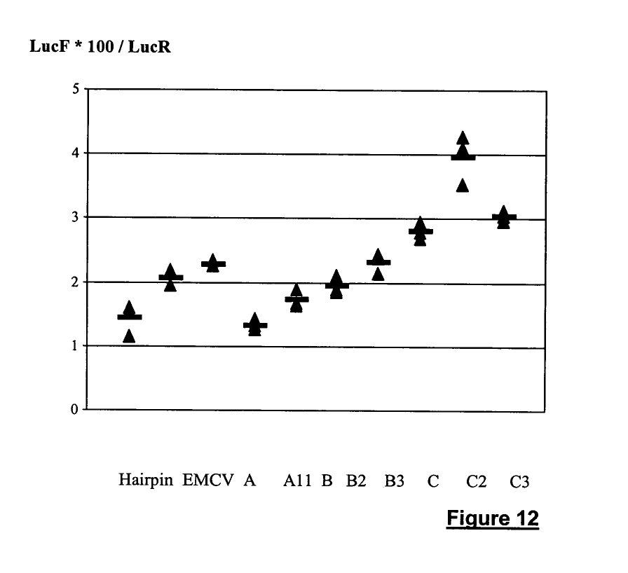

FIG. 12: Diagram comparing the IRES activity of the transcript A of human FGF-1 and of its subfragments A11, of the transcript B of human FGF-1 and of its subfragments B2 and B3, and of the transcript C of human FGF-1 and of its fragments C2 and C3, with regard to the IRES of EMCV.

### EXAMPLE 1

### IRES Activity of the 4 Transcripts A, B, C and D of Human and Murine FGF-1 Ex Vivo

A/Materials and Methods

mRNA extraction: The RNA extractions were carried out, for all the cell types, on dishes 10 cm in diameter, at confluency. Each dish was washed twice with sterile PBS and then treated with 1 ml of the RNAble lysis solution (Eurobio). The total RNAs were subsequently subjected to chloroform treatment, isopropanol precipitation and washing with 80% ethanol. After drying, they were taken up in 50 μl of water and assayed at 260 nm.

Reverse transcription: The reverse transcription reactions were carried out according to the superscript II protocol (Gibco BRL) using, for all the cell types, 2 μg of the total RNA extracts per reaction. For synthesizing the human FGF-1 cDNAs, the degenerate hexameric primers were used. For the mouse cDNAs, the 3′ primer specific for mouse FGF-1 was used. Oligonucleotides: They are presented in the 5′ to 3′ direction, the SpeI and NcoI cloning sites are underlined in the 3′ and 5′ primers, respectively. The 3′ primer is common to all the 5′NTRs of the same organism, due to its pairing at the ATG of exon 1.

Human FGF-1 Primer

3′ primer:

SEQ ID 43

CTGCTGAGCCATGGCTGAAGGG;
5′ primer A:

SEQ ID 44

ACAACTAGTGGGCATACCAGTGTCAGCTGCA;
B:

SEQ ID 45

CTAACTAGTAGAGAGCCGGGCTACTCTGAGAA;
C:

SEQ ID 46

ACAACTAGTCCAGGCAGTGGGGCCGGCTGGCCAGACTCTTGGGGG
ATTCCTTAG;
D:

SEQ ID 47

ACAACTAGTAGAGAGAGAGAAAAAATACTGTTGGCAGCAGCACAATG.

Mouse FGF-1 Primer

| | |
|---|---|
| 3′s primer: | |
| ACCACCGCTGCTTGCTGCCGAGCCATGGCTGAA; | SEQ ID 48 |
| 5′sA primer: | |
| CTCTACTAGTGTCCTTTAGTGCCAGCTAGCCTTACA; | SEQ ID 49 |
| sB: | |
| TCTCCAACTAGTAGAGCTGCAGAAATCCTGAGG. | SEQ ID 50 |

PCR: The PCRs were carried out with the platinum TAQ polymerase (Gibco BRL) for its abilities to amplify GC-rich DNA and its very good specificity. For each PCR, 3 μl of the cDNAs produced above were used. For the PCRs aimed at amplifying the 5′NTRs C and D, 50 ng of a fragment of plasmid carrying a portion of these various 5′NTRs, provided by I-M Chiu (11), were added. The murine transcripts A and B were amplified in the same way. The amplifications were carried out according to the “touchdown PCR” method. The 5′NTR A was amplified with the 5′ A primer and the 3′ primer, under the following conditions: 94° C. for 5 min, and then 3 cycles of 94° C. to 30 sec/76° C. for 2 min, and then 3 cycles of 94° C. for 30 sec/74° C. for 2 min, and then 25 cycles of 94° C. for 30 sec/72° C. for 2 min, and finally 72° C. for 5 min.

The 5′NTR B was amplified under the following conditions: 94° C. for 5 min, then 3 cycles of 94° C. for 30 sec/68° C. for 2 min, then 3 cycles of 94° C. for 30 sec/66° C. for 1 min/68° C. for 1 min, then 25 cycles of 94° C. for 30 sec/64° C. for 1 min/68° C. for 1 min, and finally 68° C. for 5 min, using the 3′ and 5′ B primers. Similarly, the 5′NTRs C and D, and the murine 5′NTRs A and B were amplified, with their corresponding primers, under conditions similar to those for the 5′NTR B, with hybridization temperatures of 70° C., 70° C., 68° C. and 66° C., respectively.

Cell culture: The cell lines were obtained from the “American Type Culture Collection” (ATCC) or the “European Collection of Cell Cultures” (ECACC), and were cultured according to their instructions.

| | | ATCC or |
|---|---|---|
| Name | Origin | ECACC no. |
| C2C12 | murine myoblast-derived line | 91031101 |
| Cos-7 | CV-1 transformed with the SV-40 | CRL-1654 |
| | virus T antigen | |
| Hela | human uterine carcinoma epithelial | CCL-2 |
| | cells | |
| L929 | immunodepressed mouse connective | CCL-1 |
| | tissues | |
| NIH-3T3 | murine embryonic fibroblast cells | CRL1658 |
| Saos-2 | human osteosarcoma epithelial cells | HTB-85 |
| | (p53 −/−) | |
| SK-Hep-1 | human liver adenocarcinoma | HTB-52 |
| SK-N-AS | human neuroblastoma cells | 94092302 |
| SK-N-BE | human neuroblastoma cells | 95011815 |

Construction of plasmids: The insertion of the various FGF-1 5′NTRs into the bicistronic vectors was carried out using the SpeI and NcoI sites located in the 5′ and 3′ PCR primers, respectively. The various plasmids were obtained by conventional molecular biology techniques, i.e.: ligation, transformation, selection of positive clones, sequencing and preparation of a concentrated solution at 1 μg/μl.

Transfections: All the transfections were carried out with the FUGENE 6 reagent according to the protocol provided by Boeringher-Roche, in a proportion of 2.5 μl per μg of plasmid DNA. The transfections for luciferase assays were carried out in 12-well dishes with 80 000 cells and 1 μg of plasmid per well.

The transfections carried out for Western blotting were performed with 2 μg of plasmid DNA in dishes 5 cm in diameter, seeded with 200 000 Cos-7 cells.

Luciferase assay: The LucR and LucF activity was measured using the “dual luciferase reporter assay” system (Promega). The transfected cells, plated out in 12-well dishes, were rinsed twice with PBS. They were then detached and homogenized in 100 μl of lysis buffer provided with the kit, 48 hours after transfection. The chemiluminescent LucF and LucR signals were measured in a luminometer (Berthold) equipped with automatic injectors.

Western blotting: 48 hours after transfection, the Cos-7 cells were washed twice with PBS and harvested in 1 ml of PBS. After centrifugation for 2 min at 2000 g, the pellet was resuspended in a 1% SDS solution containing protease inhibitors, and then lyzed by ultrasound. The proteins were assayed, with the BCA reagent (Pierce), by spectrophotometry at 595 nm. 20 μg of the proteins of each cell extract were used for the Western blotting. In summary, the cell lysates were heated at 95° C. for 2 minutes in a solution of 1% SDS, 0.1 M DTT, and loading buffer, and the proteins were then separated on a 12.5% polyacrylamide gel and transferred onto a nitrocellulose membrane. The CAT and NuCAT proteins were immunodetected by means of a rabbit polyclonal anti-CAT antibody prepared in the laboratory (1/10 000 dilution). After binding thereof, the peroxidase-coupled anti-rabbit IgG antibodies were detected by means of the chemiluminescence reagent (Amersham).

B/Results

B1/Presence of IRES Sites in the Various 5′NTRs of Human FGF-1

Cloning of the human FGF-1 5′NTR cDNA: The first step consists in cloning the various 5′NTRs of human FGF-1. To do this, the total RNAs were extracted from the human cell lines present in the laboratory. After reverse transcription, PCRs aimed at amplifying the sequences of the various FGF-1 5′NTRs were carried out. After amplification, the presence of the expected fragment indicates the existence of each transcript in the cell type tested. Amplification of the various 5′NTRs of human FGF-1 was observed from the cDNAs derived from SK-Hep-1, SK-N-BE and SK-N-AS cells. The cDNA from the SK-Hep-1 cells, which is more abundant, allowed cloning in the bicistronic vectors.

Bicistronic vectors: The bicistronic vector strategy used for demonstrating the existence of IRESs (14) was applied in order to test the IRES activity of the various FGF-1 5′NTRs in various cell types (FIG. 3). These constructs make it possible to express, from the same mRNA, two different reporter genes. The various FGF-1 5′NTRs were introduced between these two reporter genes. The first cistron provides the level of cap-dependent translation. The second cistron is expressed proportionally to the activity of internal ribosome entry in the various FGF-1 5′NTRs.

B1.1 Assay for the IRES Activity of the FGF-1 5′NTRs in Cos-7 Cells

CAT-I-NUCAT bicistronic vectors: CAT-I-NuCAT bicistronic vectors were first of all used in order to qualitatively determine, by Western blotting, the internal entry activity of the various FGF-1 5′NTRs. In fact, these vectors contain the cDNA of chloramphenicol acetyl transferase (or CAT) in the first cistron and that of a Nucleolin-CAT fusion protein (NuCAT) in the second cistron (FIG. 4). By virtue of the difference in molecular weight between these two proteins, they can be readily separated on a polyacrylamide gel under denaturing conditions. The CAT and NuCAT proteins can be readily visualized using antibodies directed against the C-terminal region of CAT, after transfer onto a membrane. Evaluation of the ratio of the second cistron to the first cistron provides information on the internal entry capacity of the inserted sequence and defines its IRES activity.

Results: The various CAT-I-NuCAT vectors, containing the FGF-1 5′NTRs, were transfected into Cos-7 cells. The Western blotting subsequent to these transfections is shown in FIG. 4.

Variations due to differences in transfection efficiency are observed in the CAT band (first cistron) However, expression of the NuCAT protein is observed in the presence of the transcripts A, B and C. These results suggest strong IRES activity of the FGF-1 variant B, and then good activity of the variant A (if it is related to the transfection efficiency, which is less in this case). The activity of the 5′NTRs C and D is much lower. Overexposure of the film also indicates an absence of alternative initiation of FGF-1 in the four 5′NTRs. This phenomenon, described and observed for FGF-2 (1), results in the formation of higher molecular weight isoforms (FIG. 4; blue arrow).

This result is in agreement with the sequence published by Chiu et al., which described the presence of a stop codon in exon 1, a few bases before the AUG of FGF-1 (11). The presence-of IRES activity for the variants A, B and C prompted a quantitative comparison of the IRES activity of these various transcripts in different cell types. For this study, since Western blotting was not a suitable technique, other bicistronic vectors encoding luciferase reporter genes were used.

B.1.2 Comparison of the IRES Activities of the FGF-1 5′NTRs in Various Cell Types

LucR-I-LucF bicistronic vectors: The LucR-I-LucF bicistronic vectors (4) express two luciferases, which use different substrates. The Renilla luciferase (LucR) is the first cistron and the Firefly luciferase (LucF) is the second cistron (FIG. 5A). LucF reports the internal entry activity and can be assayed in a very sensitive manner, like LucR. A very stable (ΔG=−40 kCal/mol) stem-loop structure was placed in a position 3′ of the LucR gene, making it possible to limit the nonspecific re-initiation phenomena observed when short sequences are introduced into the intercistronic space (FIG. 5A). The use of such a system, based on the high sensitivity of the reporter genes, allows rapid, precise and concomitant quantification of the activity of each reporter. The calculation of the LucF/LucR ratio indicates the internal entry capacity of the 5′NTR sequence and defines the IRES activity of this sequence.

The IRES activity between the various cell types is calibrated, in the laboratory, using the RHL vector (4), which contains a stem-loop structure but no IRES between the two cistrons. This vector makes it possible to determine the value for nonspecific initiation of the second cistron and to be free of the variations in transfection efficiency between the cell types.

Results: In order to study the variation in IRES activity as a function of the cell type, we chose various lines according to the expression of the four types of FGF-1 transcripts, and the results known for the FGF-2 IRES. We chose Cos-7 cells (already used with the CAT-I-NuCAT vector), a line in which it was possible to amplify the four FGF-1 5′NTRs (SK-Hep-1 cells) and a line in which the amplification did not function (Hela cells). Saos-2 cells, derived from a p53^{-/-} osteosarcoma, are the final model chosen for this study since the FGF-2 IRES is very active therein (4). The results of transfections with the. LucR-I-LucF vectors, in the Cos-7, Hela, SK-Hep-1 and Saos-2 cells, are given in FIG. 5B. It is clearly apparent, as observed in FIG. 4, that the 5′NTRs A, B and C have IRES activity. On the other hand, the activity of the 5′NTR D is very low, at the very least in the cell types tested. The activity of the 5′NTRs A, B and C is, however, very variable.

In general, it is the 5′NTR A which is the most active, the values ranging from 5.5 to 12.7. Its activity, in the Hela and Sk-Hep-1 cells, is greater than that of the FGF-2 IRES. On the other hand, the IRES of the variant A functions less well than that of FGF-2 in the Cos-7 and Saos-2 cells (although the values recorded in these cell types are the highest).

The FGF-1 variant B is more active than A in the Cos-7 cells (10 units), and its activity is moderate in the Saos-2 cells. In the other cell types tested, the 5′NTR B has no activity.

As regards the variant C, it has medium activity in the SK-Hep-1 and the Saos-2 cells (5.8 and 7.5, respectively). It is, despite everything, in the latter cell type that the 5′NTR C is the most active. Its activity in the Cos-7 and Hela cells is extremely low.

It may also be added that the information obtained, in the Cos-7 cells, with the CAT-I-NuCAT vectors can be correlated with the values obtained with the LucR-I-LucF vectors.

B2/Internal Ribosome Entry in the Various 5′NTRs of Murine FGF-1

A gene structure identical to that in humans is found in mice (FIG. 6A), bovines, pigs and chickens. Alignment of the sequences of the FGF-1 5′NTRs of these various organisms (9, 11, 16) showed good conservation of these noncoding sequences. This conservation is verified most particularly between mouse and human, with close to 70% similarity for the 5′NTR A (FIG. 7A).

The hypothesis that the IRES mechanism is conserved from mice to humans was envisioned, and we therefore carried out a comparative study of the variants A and B of human and murine FGF-1.

To do this, we amplified by RT-PCR and cloned the 5′NTRs A and B of murine FGF-1 (FIG. 6B) from murine cell line cDNA. We then used the same study strategy: construction of LucR-I-LucF bicistronic vectors, and transfection in the cell types used for the human homologue study.

Cloning of the cDNA of the 5′NTRs A and B of murine FGF-1: We used the same cloning strategy, by RT-PCR, as for the human 5′NTRs. The same amplification problems were encountered and resulted in identical enzymes and stringency conditions being used. The murine 5′NTRs were amplified and recloned, from L929 cell cDNA.

Comparison of the internal entry activity of the 5′NTRs A and B of murine and human FGF-1 in human cells: the transfections were carried out in Cos-7, Sk-Hep-1 and Saos-2 cells, in order to perform a direct comparison with the human sequences. The results subsequent to luciferase assays are given in FIG. 7B.

The IRES activity profiles for the human and murine variant A are similar. They have values of the same order of magnitude and appear to be regulated in the same manner according to the cell type, with the exception of the Saos-2 cells. In fact, the IRES activity of the murine variant A is greater in these cells and is similar to the value observed for the IRES of FGF-2. Internal entry values of the same order of magnitude are also found for the human and murine 5′NTRs B. In addition, the best IRES activity is observed in the Cos-7 cells, for the construct containing the 5′NTR B of human FGF-1: this is also the case with the murine homologue.

### EXAMPLE 2

### Characterization of the IRESs of the 4 Transcripts A, B, C and D of Human FGF-1 Ex Vivo

Example 2A: Characterization in Human Adenocarcinoma Cells (SK-Hep-1)

The characterization of the IRESs of the 4 transcripts consists in determining which is the smallest nucleotide sequence of each of the fragments A to D which has satisfactory IRES activity, which makes it possible to locate relatively precisely the IRES site of each of the transcripts. This experiment is carried out on human adenocarcinoma cells (SK-Hep-1). The following were thus tested:
the entire 5′NTR of the transcript A (A),
20 nucleotide sequences corresponding to continuous fragments of the 5′NTR of the transcript A (A2, A3, A4, A5, A6, A7, A8, A9, A10, A11, A12, A13, A14, A15, A16, A17, A18, A19, A20),
the entire 5′NTR of the transcript B (B),
4 nucleotide sequences corresponding to a continuous fragment of the 5′NTR of the transcript B (B2, B3, B4),
the entire 5′NTR of the transcript C (C),
4 nucleotide sequences corresponding to a continuous fragment of the 5′NTR of the transcript C (C2, C3, C4),
the entire 5′NTR of the transcript D (D).

The nucleotide sequences to be tested are continuous sequences derived from the messenger RNA and were subcloned into a bicistronic vector according to a procedure identical to that described in example 1. The bicistronic vector encodes two luciferases, renilla luciferase (LucR, first cistron) and firefly luciferase (LucF, second cistron). The nucleotide sequences to be tested were therefore inserted between the two luciferase genes, so as to quantify, by measuring the LucF activity, their ability to generate translation initiation by internal ribosome entry. The value of the LucF activity is related to the value of LucR activity which is, itself, proportional to the amount of mRNA. In order to express the values of IRES activity in arbitrary units (AUs), the LucF/LucR ratio is itself related to the value of a negative control (bicistronic vector without IRES) which represents the degree of “leakage”. The LucR and LucF luciferase activity was measured 48 h after transfection, on the human adenocarcinoma (SK-Hep-1) cell extracts.

The results are represented in FIGS. 8A and 8B.

IRES FGF-1A: as shown in FIG. 8A, the 5′NTR A of the FGF-1 mRNA (nucleotides 1 to 435) allows internal ribosome entry with an efficiency of approximately 14 AU (FIG. 8A, construct A), which therefore confirms the results of example 1. Analysis of various segments of this 5′NTR makes it possible to conclude that the internal entry activity does not require nt 1 to 270, nor nt 387 to 435. On the basis of this mapping, it may be concluded that the minimum IRES of FGF-1A comprises nucleotides 269 to 366 (construct A18). The IRES activity is optimized by the presence of nt located on either side, i.e. from 214 to 269 and from 366 to 435 (construct A5). It should be noted that an essential, but not necessarily sufficient, element is located between nt 270 and 304.

IRES FGF-1B: FIG. 8B indicates that the 5′NTR B of the FGF-1 mRNA (nt 1 to 147) has relatively low IRES activity, of the order of 3 AU, which therefore confirms the results of example 1. Deletion of nt 0 to 39 completely inactivates the IRES (FIG. 8B, constructs. B3 and B4).

Other examples will indicate that the IRES of FGF-1B has greater activity in other cell types.

IRES FGF-1C: FIG. 8B indicates that the 5′NTR C of the FGF-1 mRNA (149 nt) has strong IRES activity, with an efficiency of 6 AU. Interestingly, deletion of nt 103 to 149 (corresponding to the region common to the 4 NTRs of the FGF-1 mRNA) engenders an increase in the activity of the IRES (4 AU): it may therefore be concluded that the minimum IRES of FGF-1C comprises nt 1 to 103. Moreover, deletion of the first 59 nt inactivates the IRES activity.

IRES FGF-1D: FIG. 8B indicates that the 5′NTR D of the FGF-1 mRNA has no IRES activity in the SK-Hep-1 cells. However, other examples will show that this fragment has IRES activity in other cell types, in particular C2/7 myoblasts.

Example 2B: Characterization in 911 Cells (Human Embryonic Retinoblast Cells) and in C2/7 Cells (Murine Myoblast Cells)

Example 2A is repeated with the fragments A2, A6, A7 and A9.

The embryonic retinoblast cells and the myoblasts are seeded one to two days before transfection, in a 24-well plate. All the retinoblast and myoblast cell transfections were carried out with the Lipofectamine reagent according to the protocol provided by life technologies, in a proportion of 3 μl per μg of plasmid DNA per well. The cells were lysed, 48 h post-transfection, with 100 μl of lysis buffer (dual luciferase, Promega). The luciferase activities (Renilla and Firefly) were measured on 20 μl of cell lysate (mediator PhL, luminometer). for 10 s after the addition of 50 μl of reagent (dual luciferase, Promega).

The results are represented in FIGS. 9A and 9B. The hairpin is a reference sequence with a stem-loop structure, containing no IRES.

As shown in FIG. 9A, the 5′NTR of the transcript A, and also the fragments A2, A6, A7 and A9, have satisfactory IRES activity. The same is true regarding the 5′NTRs of the transcripts B, C and D in the myoblasts, whereas these same sequences exhibited less activity in the human adenocarcinoma cells (SK-Hep-1) (see example 2A).

Example 2C: Characterization in C2/7 Myoblast Cells Subsequently Differentiated into Myotubes

Example 2A is repeated with the transcripts A, A9, B, C and D, and this activity is compared with that of EMCV.

The myoblast cells are seeded one to two days before transfection, in a 12-well plate. The transfections, carried but on the fragments containing the hairpin, EMCV, A, A9, B, C or. D, are performed in duplicate as described above. The cells are lysed, 24 h post-transfection (FIG. 9C, hatched) or 9 days post-transfection (FIG. 9C, dotted). During the 9 days of incubation, the myoblast cells are differentiated into myotubes in a DMEM medium supplemented with 5% of horse serum.

As shown in FIG. 9C, the IRES activity of the FGF-1 transcript A, of the fragment A9 and of the FGF-1 transcript B is greater in the myotubes than in the myoblasts. The FGF-1 transcripts C and D have comparable activity in the myoblasts and myotubes.

It is very interesting to note that the activity, in the myotubes versus myoblasts, of the IRES of the FGF-1 transcript A (ratio=8.47), of the fragment A9 (ratio=11.63) and of the FGF-1 transcript B (ratio=18.27) is much greater than that of EMCV (ratio=6.44).

Example 2D: Characterization of Mutant Transcripts in 911 Cells (Human Embryonic Retinoblast Cells)

Example 2A is repeated using various transcripts to which are added a given sequence between the IRES° and the AUG codon of the firefly gene.

The fragments obtained are as follows:
A26: SEQ ID 26 (nt 269 to 435 of the sequence SEQ ID 8+20 nt)
A21: SEQ ID 21 (nt 269 to 435 of the sequence SEQ ID 8+40 nt)
A22: SEQ ID 22 (nt-269 to 435 of the sequence SEQ ID 8+70 nt)
A25: SEQ ID 25 (nt 269 to 435 of the sequence SEQ ID 8+135 nt)
A23: SEQ ID 23 (nt 269 to 390 of the sequence SEQ ID 9+70 nt)
A24: SEQ ID 24 (nt 269 to 390 of the sequence SEQ ID 9+135 nt)
B10: SEQ ID 34 (nt 1 to 147 of the sequence SEQ ID 30+40 nt).
B11: SEQ ID 35 (nt 1 to 147 of the sequence SEQ ID 30+70 nt)
C10: SEQ ID 40 (nt 1 to 149 of the sequence SEQ ID 36+40 nt)
C11: SEQ ID 41 (nt 1 to 149 of the sequence SEQ ID 36+70 nt)

The results are represented in FIGS. 8C and 8D.

The efficiency of the IRES of FGF1A (FIG. 8C) is increased for the mutants A26 and A21 and slightly decreased for A22 and A25. However, the segment included between nucleotides 390 and 435 is necessary in order to have IRES efficiency (mutant A23 and A24). It is observed that the efficiency of the IRES of FGF1B and 1C is increased by 4 to 6 AU relative to the wild-type IRES (FIG. 8D). Good plasticity of the fragments A, B and C is, moreover, observed.

Example 2E: Characterization of Mutant Transcripts in 911 Cells (Human Embryonic Retinoblast Cells)

Example 2A is repeated using various transcripts of the fragment A8 to which is added a sequence of 40 nt that is different from one fragment to the other, between the IRES° and the AUG codon of the firefly gene.

The fragments obtained are as follows:
A27: SEQ ID 27 (nt 269 to 435 of the sequence SEQ ID 8+40 nt)
A28: SEQ ID 28 (nt 269 to 435 of the sequence SEQ ID 8+40 nt)
A29: SEQ ID 29 (nt 269 to 435 of the sequence SEQ ID 8+40 nt).

In the three cases, satisfactory IRES activity is obtained, which again demonstrates the good plasticity of the fragment A. On about one hundred strains tested, it is observed that at least 80% of them have an efficiency equivalent to or greater than the transcript A.

### EXAMPLE 3

### Activity of the 5′NTRs of the Transcripts A, B, C and D of Human FGF-1 In Vivo

The experiments are carried out according to the following protocol.

The animals used are approximately 6-week weaned female mice. The strain used is Balb/C (IFFA CREDO). The mice are anaesthetized with a mixture of ketamine (100 mg/kg) and xylazine (10 mg/kg), intraperitoneally. The anesthesia lasts 20 minutes.

The DNA is amplified and purified by maxi preparation in the absence of endotoxin (Endofree, Qiagen). After precipitation, the DNA is taken up in a 150 mM NaCl solution (Sigma) at a concentration of approximately 50 μg and 30 μl.

The DNA is injected into the left anterior tibialis using a Hamilton syringe. The limb, which has been shaved beforehand, is coated with conducting gel. The two electrodes are applied on either side of the limb in order to effect the electrotransfer. As a general rule, 8 electric pulses of 20 milliseconds are delivered at a frequency of 2 Hz, the voltage being 200 V/cm. These experiments are carried out on groups of three animals per plasmid.

One week later, the animals are sacrificed by intraperitoneal injection of 0.1 ml of 6% sodium pentobarbital and then by cervical dislocation. The anterior tibialis is removed and frozen in liquid nitrogen. The muscles are then ground in 500 μl of lysis buffer to which a protein inhibitor cocktail has been added at 16.7 μl/ml. The muscle lysates are thawed for 5 minutes at 37° C., vortexed and centrifuged at 4° C. for 5 minutes at 15 000 G. The luciferase activities (Renilla and Firefly) are measured on 20 μl of muscle lysate (mediators PhL, luminometer) for 10 s after the addition of 50 μl of reagent (dual luciferase, Promega).

The control muscle is either a muscle which has been injected with 30 μl of 150 mM NaCl and then electroporated, or a muscle which has undergone nothing.

Example 3A: Comparison of the IRES Activity of the Transcript A of Human FGF-1 with Regard to Known IRESs

This example aims to compare, in vivo, in mouse muscle, the IRES activity of the transcript A of human FGF-1 with respect to other IRESs such as that of EMCV, VEGF, FGF2, Bip and PDGF.

As shown in FIG. 10, the activity of the IRES of FGF-1A is equivalent to that of EMCV and greater than the IRESs of VEGF, FGF-2, Bip and PDGF.

Example 3B: Comparison of the IRES Activity of the Transcript A of Human FGF-1 and of its Subfragments A, A2, A6, A7 and A9, in vivo, with Regard to the IRES of EMCV

As shown in FIG. 11, the IRES of FGF-1A and those which are modified, A2, A6 and A7, have an IRES activity similar to that of the IRES of EMCV, whereas the minimum IRES of FGF-1A (construct A9) has a slightly greater activity.

Example 3C: Comparison of the IRES Activity of the Transcript A of Human FGF-1 and of its Subfragment A11, of the Transcript B of Human FGF-1 and of its Subfragments B2 and B3, and of the Transcript C of Human FGF-1 and of its Fragments C2 and C3, with Regard to the IRES of EMCV

As shown in FIG. 12, the IRES of FGF-1B and FGF-1C has an activity similar to that of EMCV. The minimum IRES of FGF-1B (fragment B3) and of FGF-1C (fragment C2) have much greater activity than that of EMCV.

The fragment All has no activity, which confirms that the minimum IRES of FGF-1A is the fragment A9 (example 3B).

### BIBLIOGRAPHY

1. Arnaud, E., C. Touriol, C. Boutonnet, M. C. Gensac, S. Vagner, B. Prats, and A. C. Prats. 1999. A new 34-kilodalton isoform of human fibroblast growth factor 2 is cap dependently synthesized by using a non-AUG start codon and behaves as a survival factor Mol Cell Biol 19:505-14.
2. Bernstein, J., O. Sella, S. Y. Le, and O. Elry-Stein. 1997. PDGF2/c-sis mRNA leader contains a differentiation-linked internal ribosomal entry site (D-IRES). J Biol Chem 272: 9356-62.
3. Chetouani, F., P. Monestie, P. Thebault, C. Gaspin, and B. Michot. 1997. ESSA: an integrated and interactive computer tool for analysing RNA secondary structure. Nucleic Acids Res 25: 3514-22.
4. Créancier, L., D. Morello, P. Mercier, and A. C. Prats. 2000. Fibroblast growth factor 2 IRES activity ex vivo and in transgenic mice reveals a stringent tissue-specific regulation. J. Cell. Biol. (In press).
5. Duconge, F., and J. J. Toulme. 1999. In vitro selection identifies key determinants for loop-loop interactions: RNA aptamers selective for the TAR RNA element of HIV-1. Rna 5:1605-14.
6. Huez, I., L. Creancier, S. Audigier, M. C. Gensac, A. C. Prats, and H. Prats. 1998. Two independent internal ribosome entry sites are involved in translation initiation of vascular endothelial growth factor mRNA. Mol Cell Biol 18:6178-90.
7. Kozak, M. 1989. Circumstances and mechanisms of inhibition of translation by secondary structure in eukaryotic mRNAs. Mol Cell Biol 9:5134-42.
8. Macejak, D. G., and P. Sarnow. 1991. Internal initiation of translation mediated by the 5′ leader of a cellular mRNA [see comments]. Nature 353:90-4.
9. Madiai, F., K. V. Hackshaw, and I. M. Chiu. 1999. Characterization of the entire transcription unit of the mouse fibroblast growth factor 1 (FGF-1) gene. Tissue-specific expression of the FGF-1A mRNA. J Biol Chem 274:11937-44.
10. Miller, D. L., S. Ortega, O. Bashayan, R. Basch, and C. Basilico. 2000. Compensation by fibroblast growth factor 1 (FGF1) does not account for the mild phenotypic defects observed in FGF2 null mice [published erratum appears in Mol Cell Biol 2000 May; 20(10):3752]. Mol Cell Biol 20: 2260-8.
11. Myers, R. L, R. A. Payson, M. A. Chotani, L. L. Deaven, and I. M. Chiu. 1993. Gene structure and differential expression of acidic fibroblast growth factor mRNA: identification and distribution of four different transcripts. Oncogene 8:341-9.
12. Nanbru, C., I. Lafon, S. Audigier, M. C. Gensac, S. Vagner, G. Huez, and A. C. Prats. 1997. Alternative translation of the proto-oncogene c-myc by an internal ribosome entry site. J Biol Chem 272:32061-6.
13. Payson, R. A., M. A. Chotani, and I. M. Chiu. 1998. Regulation of a promoter of the fibroblast growth factor 1 gene in prostate and breast cancer cells. J Steroid Biochem Mol Biol 66:93-103.
14. Pelletier, J., and N. Sonenberg. 1988. Internal initiation of translation of eukaryotic mRNA directed by a sequence derived from poliovirus RNA. Nature 334:320-5.
15. Pennisi, E. 2000. Human Genome Project. And the number of gene is . . . ? [news]. Science 288:1146-7.
16. Philippe, J. M., F. Renaud, Y. Courtois, and M. Laurent. 1996. Cloning of multiple chicken FGF1 mRNAs and their differential expression during development of whole embryo and of the lens. DNA Cell Biol 15: 703-15.
17. Pyronnet, S., L. Pradayrol, and N. Sonenberg. 2000. A Cell Cycle-Dependant Internal Ribosome Entry Site. Mol. Cell 5:607-616.
18. Vagner, S., M. C. Gensac, A. Maret, F. Bayard, F. Amalric, H. Prats, and A. C. Prats. 1995. Alternative translation of human fibroblast growth factor 2 mRNA occurs by internal entry of ribosomes. Mol Cell Biol 15:35-44.
19. Vagner, S., A. Waysbort, M. Marenda, M. C. Gensac, F. Amalric, and A. C. Prats. 1995. Alternative translation initiation of the Moloney murine leukemia virus mRNA controlled by internal ribosome entry involving the p57/PTB splicing factor. J Biol Chem 270:20376-83.
20. West, M. J., N. F. Sullivan, and A. E. Willis. 1995. Translational upregulation of the c-myc oncogene in Bloom's syndrome cell lines. Oncogene 11:2515-24.

1. The use, for its IRES activity, of an isolated nucleic acid containing a sequence identical or homologous to that of the transcripts A, B, C and D of FGF1 corresponding, respectively, to the sequences SEQ ID 1(A), SEQ ID 30 (B), SEQ ID 36 (C) and SEQ ID 42 (D).

2. The use as claimed in claim 1, characterized in that the nucleic acid contains a sequence identical or homologous to a sequence chosen from the group comprising SEQ ID 2 (A2), SEQ ID 3 (A3), SEQ ID 4 (A4), SEQ ID 5 (A5), SEQ ID 6 (A6), SEQ ID 7 (A7), SEQ ID 8 (A8), SEQ ID 9 (A9), SEQ ID 12 (A12), SEQ ID 16 (A16), SEQ ID 17 (A17), SEQ ID 18 (A18), SEQ ID 19 (A19), SEQ ID 20 (A20), SEQ ID 26 (A26), SEQ ID 21 (A21), SEQ ID 27 (A27), SEQ ID 28 (A28) and SEQ ID 29 (A29).

3. The use as claimed in claim 1, characterized in that the nucleic acid contains a sequence identical or homologous to a sequence chosen from the group comprising SEQ ID 31 (B2) and SEQ ID 34 (B10).

4. The use of an isolated nucleic acid as claimed in claim 1, characterized in that it contains a sequence identical or homologous to a sequence chosen from the group comprising SEQ ID 36 (C2), SEQ ID 40 (C10) and SEQ ID 41 (C11).

5. A cassette for expressing at least one gene of interest in a eukaryotic cell, wherein said cassette comprises at least one nucleic acid having a sequence identical or homologous to one of SEQ ID NO. 1, SEQ ID NO. 30, SEQ ID NO. 36 or SEQ ID NO. 42.

6. The expression cassette as claimed in claim 5, wherein said cassette contains at least two genes of interest.

7. A recombinant expression vector, wherein said vector comprises the expression cassette of claim 5.

8. The expression vector of claim 7, wherein said vector is a plasmid vector or a viral vector chosen from the group consisting of retroviruses, lentiviruses and adenoviruses.

9. A pharmaceutical composition comprising the expression cassette of claim 5.

10. A recombinant host cell, wherein said cell comprises the expression cassette of claim 5.

11. A transgenic animal, wherein said animal comprises the expression cassette of claim 5.

12. An isolated nucleic acid having IRES activity, consisting of a sequence exhibiting at least 60% identity with a sequence chosen from the group SEQ ID 1(A), SEQ ID 30 (B), SEQ ID 36 (C), SEQ ID 42 (D), SEQ ID 2 (A2), SEQ ID 3 (A3), SEQ ID 4 (A4), SEQ ID 5 (A5), SEQ ID 6 (A6), SEQ ID 7 (A7), SEQ ID 8 (A8), SEQ ID 9 (A9), SEQ ID 12 (A12), SEQ ID 16 (A16), SEQ ID 17 (A17), SEQ ID 18 (A18), SEQ ID 19 (A19), SEQ ID 20 (A20), SEQ ID 26 (A26), SEQ ID 21 (A21), SEQ ID 27 (A27), SEQ ID 28 (A28), SEQ ID 29 (A29), SEQ ID 31 (B2), SEQ ID 34 (B10), SEQ ID 36 (C2), SEQ ID 40 (C10), SEQ ID 41 (C11) and SEQ ID 42 (D).

13. A pharmaceutical composition comprising the recombinant vector of claim 7.

14. A recombinant host cell comprising the recombinant vector of claim 7.

15. A transgenic animal comprising the recombinant vector of claim 7.


SEQUENCE LISTING (50 sequences)

SEQ ID NO: 1 - DNA, 435 bp, artificial sequence
  5′UTR 1..435: corresponds to 5′nontranslated region of transcript A of human FGF-1
gggcataccagtgtcagctgcacttgtaggggcccaagtgcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagccatg

SEQ ID NO: 2 - DNA, 390 bp, artificial sequence
  5′UTR 1..390: corresponds to a portion of the 5′nontranslated region of transcript A of human FGF-1 having IRES activity (A2)
gggcataccagtgtcagctgcacttgtaggggcccaagtgcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaag

SEQ ID NO: 3 - DNA, 396 bp, artificial sequence
  5′UTR 39..435: corresponds to a portion of the 5′nontranslated region of transcript A of human FGF-1 having IRES activity (A3)
gcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagccatg

SEQ ID NO: 4 - DNA, 313 bp, artificial sequence
  5′UTR 122..435: corresponds to a portion of the 5′nontranslated region of transcript A of human FGF-1 having IRES activity (A4)
gtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagccatg

SEQ ID NO: 5 - DNA, 221 bp, artificial sequence
  5′UTR 214..435: corresponds to a portion of the 5′nontranslated region of transcript A of human FGF-1 having IRES activity (A5)
gccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagccatg

SEQ ID NO: 6 - DNA, 268 bp, artificial sequence
  5′UTR 122..390: corresponds to a portion of the 5′nontranslated region of transcript A of human FGF-1 having IRES activity (A6)
gtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaag

SEQ ID NO: 7 - DNA, 176 bp, artificial sequence
  5′UTR 214..390: corresponds to a portion of the 5′nontranslated region of transcript A of human FGF-1 having IRES activity (A7)
gccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgccccagaaagaaagcaccccagagccaag

SEQ ID NO: 8 - DNA, 166 bp, artificial sequence
  5′UTR 269..435: corresponds to a portion of the 5′nontranslated region of transcript A of human FGF-1 having IRES activity (A8)
tctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagccatg

SEQ ID NO: 9 - DNA, 121 bp, artificial sequence
  5′UTR 269..390: corresponds to a portion of the 5′nontranslated region of transcript A of human FGF-1 having IRES activity (A9)
tctgatggtgaatgggaactcccttcctcctgcagcagcctgcctgcagctgtcctggtagaacagtgtggacattgcagaagctgtcactgccccagaaagaaagcaccccagagccaag

SEQ ID NO: 10 - DNA, 132 bp, artificial sequence
  5′UTR 303..435: corresponds to a portion of the 5′nontranslated region of transcript A of human FGF-1 having IRES activity (A10)
gcagcctgcctgcagctgtcctggtagaacagtgtggacattgcagaagctgtcactgccccagaaagaaagcaccccagagccaaggcaaagagtcttgaaagcgccacaagcagcagctgctgagccatg

SEQ ID NO: 11 - DNA, 86 bp, artificial sequence
  5′UTR 303..390: corresponds to a portion of the 5′nontranslated region of transcript A of human FGF-1 having IRES activity (A11)
gcagcctgcctgcagctgtcctggtagaacagtgtggacattgcagaagctgtcactgccccagaaagaaagcaccccagagccaa

SEQ ID NO: 12 - DNA, 366 bp, artificial sequence
  5′UTR 1..366: corresponds to a portion of the 5′nontranslated region of transcript A of human FGF-1 having IRES activity (A12)
gggcataccagtgtcagctgcacttgtaggggcccaagtgcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgcccca

SEQ ID NO: 13 - DNA, 153 bp, artificial sequence
  5′UTR 282..435: corresponds to a portion of the 5′nontranslated region of transcript A of human FGF-1 having IRES activity (A13)
gggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagccatg

SEQ ID NO: 14 - DNA, 108 bp, artificial sequence
  5′UTR 282..390: corresponds to a portion of the 5′nontranslated region of transcript A of human FGF-1 having IRES activity (A14)
gggaactcccttcctcctgcagcagcctgcctgcagctgtcctggtagaacagtgtggacattgcagaagctgtcactgccccagaaagaaagcaccccagagccaag

SEQ ID NO: 15 - DNA, 84 bp, artificial sequence
  5′UTR 282..366: corresponds to a portion of the 5′nontranslated region of transcript A of human FGF-1 having IRES activity (A15)
gggaactcccttcctcctgcagcagcctgcctgcagctgtcctggtagaacagtgtggacattgcagaagctgtcactgcccca

SEQ ID NO: 16 - DNA, 244 bp, artificial sequence
  5′UTR 122..366: corresponds to a portion of the 5′nontranslated region of transcript A of human FGF-1 having IRES activity (A16)
gtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgcccca

SEQ ID NO: 17 - DNA, 152 bp, artificial sequence
  5′UTR 214..366: corresponds to a portion of the 5′nontranslated region of transcript A of human FGF-1 having IRES activity (A17)
gccttcgctccaggggaatcagggcatcgcctccttttctgggaggacactcccttctgatggtgaatgggaactcccttcctcctgcagcagcctgcctgcagctgtcctggtagaacagtgtggacattgcagaagctgtcactgcccca

SEQ ID NO: 18 - DNA, 97 bp, artificial sequence
  5′UTR 269..366: corresponds to a portion of the 5′nontranslated region of transcript A of human FGF-1 having IRES activity (A18)
tctgatggtgaatgggaactcccttcctcctgcagcagcctgcctgcagctgtcctggtagaacagtgtggacattgcagaagctgtcactgcccca

SEQ ID NO: 19 - DNA, 344 bp, artificial sequence
  5′UTR 1..344: corresponds to a portion of the 5′nontranslated region of transcript A of human FGF-1 having IRES activity (A19)
gggcataccagtgtcagctgcacttgtaggggcccaagtgcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtagaacagtgtggacat

SEQ ID NO: 20 - DNA, 330 bp, artificial sequence
  5′UTR 1..320: corresponds to a portion of the 5′nontranslated region of transcript A of human FGF-1 having IRES activity (A20)
gggcataccagtgtcagctgcacttgtaggggcccaagtgcctcatgacccactcggcagccttcctctccaggatccccaaggctaggaggccaacctactaacaggtgggtgggtatggtgtgtggtttcactcagttcttctcatggggtttctctgagctccattcataccagaaagggagcaggagagagaggacaagtggatccaacagccttcgctccaggggaatcagggcatcgcctccttttctgggaggacactcccttctgatggtgaatgggaactcccttcctcctgcagcagcctgcctgcagctgtcctggtag

SEQ ID NO: 21 - DNA, 205 bp, artificial sequence
  5′UTR 1..205: corresponds to nt269 to 435 of the 5′nontranslated region of transcript A of human FGF-1 plus 40 nt (A21)
tctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgaggacttgaagtcagctgagggatttccgtaagcatcgtgaccatg

SEQ ID NO: 22 - DNA, 235 bp, artificial sequence
  5′UTR 1..235: corresponds to nt269 to 435 of the 5′nontranslated region of transcript A of human FGF-1 plus 70 nt (A22)
tctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgaggacttgaagtcagctgagggatttccgtaagcatcgtgatcgattagccttaagccttgaatccgagtcccatg

SEQ ID NO: 23 - DNA, 192 bp, artificial sequence
  5′UTR 1..192: corresponds to nt269 to 390 of the 5′nontranslated region of transcript A of human FGF-1 plus 70 nt (A23)
tctgatggtgaatgggaactcccttcctcctgcagcagcctgcctgcagctgtcctggtagaacagtgtggacattgcagaagctgtcactgccccagaaagaaagcaccccagagccgacttgaagtcagctgagggatttccgtaagcatcgtgatcgattagccttaagccttgaatccgagtcccatg

SEQ ID NO: 24 - DNA, 257 bp, artificial sequence
  5′UTR 1..257: corresponds to nt269 to 390 of the 5′nontranslated region of transcript A of human FGF-1 plus 135 nt (A24)
tctgatggtgaatgggaactcccttcctcctgcagcagcctgcctgcagctgtcctggtagaacagtgtggacattgcagaagctgtcactgccccagaaagaaagcaccccagagccgacttgaagtcagctgagggatttccgtaagcatcgtgatcgattagccttaagccttgaatccgagtcgggattagcggccgcattaatgactatcgcacacatctggaaccatgcgtggatccaatgtcccgccatg

SEQ ID NO: 25 - DNA, 300 bp, artificial sequence
  5′UTR 1..300: corresponds to nt269 to 435 of the 5′nontranslated region of transcript A of human FGF-1 plus 135 nt (A25)
tctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgaggacttgaagtcagctgagggatttccgtaagcatcgtgatcgattagccttaagccttgaatccgagtcgggattagcggccgcattaatgactatcgcacacatctggaaccatgcgtggatccaatgtcccgccatg

SEQ ID NO: 26 - DNA, 185 bp, artificial sequence
  5′UTR 1..185: corresponds to nt269 to 435 of the 5′nontranslated region of transcript A of human FGF-1 plus 20 nt (A26)
tctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgaggacttgaagtcagctgaggccatg

SEQ ID NO: 27 - DNA, 206 bp, artificial sequence
  5′UTR 1..206: corresponds to nt269 to 435 of the 5′nontranslated region of transcript A of human FGF-1 (SEQ ID NO.8) plus 40 nt (A27)
tctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagggggttacagatgctggagatacgtgggcaaccttggggaccatg

SEQ ID NO: 28 - DNA, 206 bp, artificial sequence
  5′UTR 1..206: corresponds to nt269 to 435 of the 5′nontranslated region of transcript A of human FGF-1 (SEQ ID NO.8) plus 40 nt (A28)
tctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgaggagcttgcagatcacctgccccgggttgggagtcctctacccatg

SEQ ID NO: 29 - DNA, 206 bp, artificial sequence
  5′UTR 1..206: corresponds to nt269 to 435 of the 5′nontranslated region of transcript A of human FGF-1 (SEQ ID NO.8) plus 40 nt (A29)
tctgatggtgaatgggaactcccttcctcctgcagcagcctgcctgcagctgtcctggtagaacagtgtggacattgcagaagctgtcactgccccagaaagaaagcaccccagagccaaggcaaagagtcttgaaagcgccacaagcagcagctgctgagagtggcgagttcattcagtgagctccccgttggtcttgcaccatg

SEQ ID NO: 30 - DNA, 147 bp, artificial sequence
  5′UTR 1..147: corresponds to the 5′nontranslated region of transcript B of human FGF-1 having IRES activity (B)
agagagccgggctactctgagaagaagacaccaagtggattctgcttcccctgggacagcactgagcgagtgtggagagaggtacagccctcggcctacaagctctttagtcttgaaagcgccacaagcagcagctgctgagccatg

SEQ ID NO: 31 - DNA, 93 bp, artificial sequence
  5′UTR 1..93: corresponds to a portion of the 5′nontranslated region of transcript B of human FGF-1 having IRES activity (B2)
agagagccgggctactctgagaagaagacaccaagtggattctgcttcccctgggacagcactgagcgagtgtggagagaggtacagccctcg

SEQ ID NO: 32 - DNA, 108 bp, artificial sequence
  5′UTR 39..147: corresponds to a portion of the 5′nontranslated region of transcript B of human FGF-1 having IRES activity (B3)
ttctgcttcccctgggacagcactgagcgagtgtggagagaggtacagccctcggcctacaagctctttagtcttgaaagcgccacaagcagcagctgctgagccatg

SEQ ID NO: 33 - DNA, 54 bp, artificial sequence
  5′UTR 39..93: corresponds to a portion of the 5′nontranslated region of transcript B of human FGF-1 having IRES activity (B4)
ttctgcttcccctgggacagcactgagcgagtgtggagagaggtacagccctcg

SEQ ID NO: 34 - DNA, 186 bp, artificial sequence
  5′UTR 1..186: corresponds to nt1 to 147 of the 5′nontranslated region of transcript B of human FGF-1 (SEQ ID NO.30) plus 40 nt (B10)
agagagccgggctactctgagaagaagacaccaagtggattctgcttcccctgggacagcactgagcgagtgtggagagaggtacagccctcggcctacaagctctttagtcttgaaagcgccacaagcagcagctgctgaggacttgaagtcagctgagggatttccgtaagcatcgtgaccatg

SEQ ID NO: 35 - DNA, 216 bp, artificial sequence
  5′UTR 1..216: corresponds to nt1 to 147 of the 5′nontranslated region of transcript B of human FGF-1 (SEQ ID NO.30) plus 40 nt (B11)
agagagccgggctactctgagaagaagacaccaagtggattctgcttcccctgggacagcactgagcgagtgtggagagaggtacagccctcggcctacaagctctttagtcttgaaagcgccacaagcagcagctgctgaggacttgaagtcagctgagggatttccgtaagcatcgtgatcgattagccttaagccttgaatccgagtcccatg

SEQ ID NO: 36 - DNA, 149 bp, artificial sequence
  5′UTR 1..149: corresponds to the 5′nontranslated region of transcript C of human FGF-1 having IRES activity (C)
ccaggcagtggggccggctggccagactcttgggggattccttagtgagtgagttcactgctcaaagaagggctttgccacttctgcagggaagccagccacgggccagcagtcttgaaagcgccacaagcagcagctgctgagccatg

SEQ ID NO: 37 - DNA, 103 bp, artificial sequence
  5′UTR 1..103: corresponds to a portion of the 5′nontranslated region of transcript C of human FGF-1 having IRES activity (C2)
ccaggcagtggggccggctggccagactcttgggggattccttagtgagtgagttcactgctcaaagaagggctttgccacttctgcagggaagccagccacg

SEQ ID NO: 38 - DNA, 90 bp, artificial sequence
  5′UTR 59..149: corresponds to a portion of the 5′nontranslated region of transcript C of human FGF-1 having IRES activity (C3)
gctcaaagaagggctttgccacttctgcagggaagccagccacgggccagcagtcttgaaagcgccacaagcagcagctgctgagccatg

SEQ ID NO: 39 - DNA, 45 bp, artificial sequence
  5′UTR 59..103: corresponds to a portion of the 5′nontranslated region of transcript C of human FGF-1 having IRES activity (C4)
tgctcaaagaagggctttgccacttctgcagggaagccagccacg

SEQ ID NO: 40 - DNA, 189 bp, artificial sequence
  5′UTR 1..189: corresponds to nt1 to 149 of the 5′nontranslated region of transcript C of human FGF-1 (SEQ ID NO. 36)plus 40 nt (C10)
cccaggcagtggggccggctggccagactcttgggggattccttagtgagtgagttcactgctcaaagaagggctttgccacttctgcagggaagccagccacgggccagcagtcttgaaagcgccacaagcagcagctgctgaggacttgaagtcagctgagggatttccgtaagcatcgtgaccatg

SEQ ID NO: 41 - DNA, 218 bp, artificial sequence
  5′UTR 1..189: corresponds to nt1 to 149 of the 5′nontranslated region of transcript C of human FGF-1 (SEQ ID NO. 36)plus 70 nt (C11)
ccaggcagtggggccggctggccagactcttgggggattccttagtgagtgagttcactgctcaaagaagggctttgccacttctgcagggaagccagccacgggccagcagtcttgaaagcgccacaagcagcagctgctgaggacttgaagtcagctgagggatttccgtaagcatcgtgatcgattagccttaagccttgaatccgagtcccatg

SEQ ID NO: 42 - DNA, 92 bp, artificial sequence
  5′UTR 1..92: corresponds to the 5′nontranslated region of transcript D of human FGF-1 having IRES activity (D)
agagagagagaaaaaatactgttggcagcagcacaatgtttgggctaagacctggtcttgaaagcgccacaagcagcagctgctgagccatg

SEQ ID NO: 43 - DNA, 22 bp, artificial Sequence
  primer_bind 1..22: human FGF1 3′primer
ctgctgagccatggctgaaggg

SEQ ID NO: 44 - DNA, 31 bp, artificial sequence
  primer_bind 1..31: 5′primer to human FGF1 transcript A
acaactagtgggcataccagtgtcagctgca

SEQ ID NO: 45 - DNA, 32 bp, artificial Sequence
  primer_bind 1..32: 5′ primer to human FGF1 transcript B
ctaactagtagagagccgggctactctgagaa

SEQ ID NO: 46 - DNA, 54 bp, artificial Sequence
  primer_bind 1..54: 5′primer to human FGF1 transcript C
acaactagtccaggcagtggggccggctggccagactcttgggggattccttag

SEQ ID NO: 47 - DNA, 47 bp, artificial sequence
  primer_bind 1..47: 5′primer to human FGF1 transcript D
acaactagtagagagagagaaaaaatactgttggcagcagcacaatg

SEQ ID NO: 48 - DNA, 33 bp, Artificial sequence
  primer_bind 1..33: 3′s primer to mouse FGF1
accaccgctgcttgctgccgagccatggctgaa

SEQ ID NO: 49 - DNA, 36 bp, Artificial sequence
  primer_bind 1..36: 5′ sA primer to mouse FGF1
ctctactagtgtcctttagtgccagctagccttaca

SEQ ID NO: 50 - DNA, 33 bp, artificial Sequence
  primer_bind 1..31: sB primer to mouse FGF1
tctccaactagtagagctgcagaaatcctgagg